(19) United States
(12) Patent Application Publication
(10) Pub. No.: US 2024/0101993 A1
(43) Pub. Date: Mar. 28, 2024

(54) ENHANCING EFFICIENCY OF TARGETED GENE KNOCKIN BY BASE EDITORS

(71) Applicant: REGENTS OF THE UNIVERSITY OF MINNESOTA, Minneapolis, MN (US)

(72) Inventors: Beau R. Webber, Minneapolis, MN (US); Branden S. Moriarity, Minneapolis, MN (US); Walker Lahr, Minneapolis, MN (US); Nicholas Slipek, Minneapolis, MN (US); Mitchell G. Kluesner, Seattle, WA (US)

(73) Assignee: REGENTS OF THE UNIVERSITY OF MINNESOTA, Minneapolis, MN (US)

(21) Appl. No.: 18/273,454

(22) PCT Filed: Jan. 21, 2022
(86) PCT No.: PCT/US22/13390

§ 371 Date: Jul. 20, 2023

Related U.S. Application Data
(60) Provisional application No. 63/140,032, filed on Jan. 21, 2021.

(51) Int. Cl. C12N 9/78; C12N 9/22; C12N 15/11; C12N 15/90

(52) U.S. Cl. CPC C12N 9/78; C12N 9/22; C12N 15/111; C12N 15/907; C12Y 305/04002; C12N 2310/20; C12N 2800/80

(57) ABSTRACT

The present invention provides genomic engineering methods that utilize base editors to perform targeted gene knockins and gene knockouts with high efficiency. Cells that have been genetically modified according to these methods are also provided, as are methods of using the modified cells in a treatment for cancer.

### CROSS REFERENCE TO RELATED APPLICATIONS

This application claims the benefit of priority under 35 U.S.C. § 119(e) of U.S. Provisional Application No. 63/140,032, filed Jan. 21, 2021, the contents of which is incorporated herein by reference in its entirety.

### STATEMENT REGARDING FEDERALLY SPONSORED RESEARCH

N/A

### SEQUENCE LISTING

A Sequence Listing accompanies this application and is submitted as an ASCII text file of the sequence listing named “920171_00453_ST25.txt” which is 12,852 bytes in size and was created on Jan. 21, 2022. The sequence listing is electronically submitted via EFS-Web with the application and is incorporated herein by reference in its entirety.

### BACKGROUND

Genetic engineering of patient cells is an attractive route for therapy due to the permanency of treatment and the low risk of rejection by the patient. Clustered regularly interspaced short palindromic repeats (CRISPR)-based gene editing is one powerful tool that can be used to perform genetic engineering CRISPR systems are used to induce a double-strand break (DSB) within a gene of interest, which often results in the formation of small insertions or deletions (collectively referred to as ‘indels’) by the non-homologous end joining (NHEJ) pathway. Alternatively, precise genomic alterations can be achieved by introducing a DSB while co-delivering a DNA donor template, from which the cell can repair the DSB via homology directed repair (HDR). While this approach is efficient and reliable, inducing multiple DSBs during multiplexed gene editing procedures can cause undesirable genotoxicity and the formation of potentially oncogenic gross chromosomal translocations. Accordingly, there remains a need in the art for more controlled and safer methods of multiplexed genetic engineering that cause limited induction of toxic DSBs.

### SUMMARY

In a first aspect, the disclosure provides methods for producing genetically modified cells.

In one embodiment, the methods comprise introducing into the cells: (i) a plasmid, mRNA, or protein encoding a base editor; (ii) two primary gRNAs that are complementary to the 5′ and 3′ ends of a knockin target site prior to editing; (iii) two retargeting gRNAs that are complementary to the 5′ and 3′ ends of a knockin target site after they have been edited by complexes comprising the base editor and the primary gRNAs; and (iv) a DNA donor template; thereby generating cells with a targeted knockin via homology directed repair from the DNA donor template.

In a second embodiment, the methods comprise introducing into the cells: (i) a plasmid, mRNA, or protein encoding a base editor; (ii) at least two primary gRNAs that are complementary to the 5′ and 3′ ends of a knockin target site prior to editing; and (iii) a DNA donor template; thereby generating cells with a targeted knockin via homology directed repair from the DNA donor template; and wherein the rate of indel formation at the knockin target site is less than 10%

In some embodiments, the methods further comprise introducing into the cells: one or more primary gRNAs that are complementary to a knockout target site, wherein the method generates cells with at least one targeted knockin and at least one targeted knockout. In some embodiments, the methods further comprise introducing into the cells at least one additional set of reagents that target a different knockin target site.

In a second aspect, the disclosure provides genetically modified cell obtained according to a method described herein.

In third aspect, the disclosure provides a use of a genetically modified cell described herein for the treatment of cancer.

### BRIEF DESCRIPTION OF THE DRAWINGS

FIG. 1 demonstrates that a multi-enzyme system comprising base editor and an orthologous nickase can be used to perform gene knockouts and gene knockins simultaneously. (A) A schematic depiction of this multi-enzyme system, wherein a base editor is used to perform gene knockouts while an orthologous nuclease is used to perform targeted knockins. The orthologous nuclease creates a double-strand break that is repaired from a DNA donor template encoding GFP, which is delivered using an adeno-associated virus (AAV) vector. (B) The genes TRAC, PD1, B2M, and CISH were knocked out from primary human T cells using the multi-enzyme system. The height of the bars represents the average rate of protein loss for the indicated target genes. A digital western blot shows representative image of CISH protein loss. N=2 biological replicates using different DNA donor templates. (C) Rates of GFP knockin in primary human T cells treated with the multi-enzyme system. N=2 biological replicates using different DNA donor templates.

FIG. 2 demonstrates that a single-enzyme system comprising only a base editor can be used to perform simultaneous gene knockouts and gene knockins, but with low efficiency. (A) A schematic depiction of this single-enzyme system, wherein the base editor is used to perform both the gene knockouts and gene knockins. In this system, two independent nicking gRNAs are used to integrate a splice acceptor GFP cassette from a donor template. (B) Efficiencies of targeted GFP integration using various cytosine base editors (CBEs) expressed under the control of the endogenous adeno-associated virus integration site 1 (AAVS1) promoter in primary human T cells. GFP-positive cells were quantified using flow cytometry.

FIG. 3 illustrates a proposed mechanism by which Cas9 nickase generates knockins without retargeting gRNAs. Initial states, Cas9-bound states, desired states, and editing sinks are each highlighted in colored boxes. The term “editing sinks” refers to allelic states that are unable to enter the editing circuit. Arrows connecting states show directionality as well as some rate (“K”) dictating the speed of that transition. Note that the initial allelic state (blue) can be restored by double-strand breaks through homology directed repair.

FIG. 4 illustrates a proposed mechanism by which adenine base editors (ABEs) generate knockins without retargeting gRNAs. Initial states, ABE-bound states, desired states, and editing sinks are highlighted in colored boxes. Note that editing sinks containing I:C base pairs resulting from editing of initial A:T base pairs are unable to participate further in the circuit. However, the introduction of retargeting gRNAs that bind to the editing sink alleles allow for these alleles to reenter the editing circuit and undergo homology directed repair, thereby increasing the rate of integration of the desired donor sequence.

FIG. 5 illustrates a proposed mechanism by which CBEs generate knockins without retargeting gRNAs. Initial states, CBE-bound states, desired states, and editing sinks are highlighted in colored boxes. Note that editing sinks containing T:A base pairs resulting from editing of initial C:G base pairs are unable to participate further in the circuit. However, the introduction of retargeting gRNAs that bind to the editing sink alleles allow for these alleles to reenter the editing circuit and undergo homology directed repair, thereby increasing the rate of integration of desired donor sequence.

FIG. 6 conceptually illustrates how the inclusion of retargeting gRNAs in the base editor method increases transgene integration. (A) When a Cas9 nickase is used for homology directed repair (HDR), the introduction of two gRNAs allows the nickase to generate a double-strand break (DSB) with single-stranded DNA (ssDNA) overhangs, which allows for transgene integration through HDR. In this system, nicks will be generated continuously in one or both strands of DNA. While HDR can occur even in the absence of nicks in the DNA backbone when a homologous sequence is in close proximity and the cellular condition are right, the rate of HDR is increased if one of the DNA backbones is nicked, and it is increased dramatically if both of the DNA backbones are nicked. After Cas9 nickase releases from the two cut sites, HDR can be used to repair the genomic DNA, either from the complementary strand of DNA or from a provided DNA donor template. This process is thought to occur repeatedly until a steady state is reached. (B) When a base editor is used for HDR, the nickase function of base editor is used to create nicks on one or both strands of DNA. However, the HDR efficiency is much lower with a base editor than with a Cas9 nickase due to the deamination of the binding site. When the base editor modifies the target sequence via its editing function, this reduces the ability of the target site to undergo continuous retargeting and re-nicking using the original gRNAs (left panel). However, including a gRNA that binds to the edited sequence (i.e., a retargeting gRNA) allows for continued re-nicking after the original bases have been edited. In other words, the retargeting gRNA are designed to allow the base editor to continue to bind to and perform its nickase function on the edited sequence generated by the deaminase function of the base editor. Once bound by the base editor and the retargeting gRNA, nicking will continue to occur for the lifetime of the base editor without the need for further retargeting gRNAs. In this way, this dual-nicking strategy drives the cell towards higher rates of HDR.

FIG. 7 demonstrates that retargeting gRNAs increase the knockin efficiency of the single-enzyme system. (A) T cells were thawed and stimulated for 48 hours. Cells were electroporated with mRNA encoding either Cas9 nickase or the adenine base editor ABE8e as well as a cocktail of dual-nicking gRNAs, with or without retargeting gRNAs. Following electroporation, cells were incubated with AAV carrying a dsDNA donor template comprising a fluorescence gene flanked by 1 kb homology arms that were designed to target the AAVS1 locus. The cells were then restimulated with Dynabeads, grown in a G-Rex® for 7 days, and analyzed by flow cytometry for GFP expression. (B) T cells were stimulated and electroporated as described above and were then incubated with AAV carrying a dsDNA donor template comprising a gene encoding a CD33-specific chimeric antigen receptor (CAR) flanked by 0.6 kb homology arms that target the AAVS1 locus. Cells were grown, stained with CD33-conjugated protein, and analyzed by flow cytometry. (C) T cells were stimulated and electroporated, as described above. The T cells were then incubated with AAV carrying a dsDNA donor template comprising a gene encoding the fluorescence protein. These data demonstrate that the inclusion of retargeting gRNAs improves the knockin efficiency of ABE8e but not coBE4. This suggest that a more complex mechanism may underlie CBE knockin, as is diagrammed in FIG. 5.

FIG. 8 shows a genomic map of the AAVS1 gRNA target sites before and after editing with a cytosine base editor (CBE). (A) The DNA sequence prior to editing. (B) The DNA sequence following CBE editing, wherein the cytosines within the gRNA editing window are converted to thymines, preventing rebinding of gRNAs.

FIG. 9 shows a schematic of AAVS1 CBE retargeting.

FIG. 10 shows a genomic map of the AAVS1 gRNA target sites before and after editing with an adenine base editor (ABE). (A) The DNA sequence prior to editing. (B) The DNA sequence following ABE editing, wherein the adenines within the gRNA editing window are converted to guanines, preventing rebinding of gRNAs.

FIG. 11 shows a schematic of AAVS1 ABE retargeting.

FIG. 12 shows that the base editor retargeting method works at loci other than AAVS1. (A) Schematic of the T-cell receptor α constant (TRAC) genomic region targeted by gRNAs for dual-nicking using ABE8e-NG. ABE8e-NG is a report an engineered SpCas9 variant that can recognize relaxed NG PAMs (see Science 361(6408):1259-1262, 2018). The left gRNAs are labeled “LN” and the right gRNAs are labeled “RN”. The pam sequence is annotated on each gRNA with a dashed line. (B) Integration rates of GFP or CAR-T2A-RQR8 AAV6 vectors delivered to primary human T cells following dual-nicking with ABE8e-NG analyzed by flow cytometry. Flow cytometry was performed with a viability dye and an antibody specific to the RQR8 molecule. GFP expression data was collected for all samples.

FIG. 13 shows an analysis of knockout efficiency and transgene expression in primary human T cells edited with ABE8e using the base editor retargeting method. (A) Diagram of gene editing strategy. Briefly, T cells were magnetically isolated from human blood and activated with CD3/CD28 beads. Cells were then electroporated with gene editing reagents and allowed to rest before the addition of transgene carrying AAV. Cells were expanded 5-10 days prior to collection for further analysis and experimentation. Upon collection, cells were stained with fluorescently labelled antibodies for flow cytometric analysis or gDNA was isolated for base conversion and indel analysis. Flow cytometry analysis showed significant knockout of the genes (B) TRAC, (D) B2M, and (F) PD1 as well as knockin of genes encoding (J) GFP, (K) Meso-CAR, and (L) CD33-CAR. The CISH KO is not shown. gDNA was isolated and amplified using Nextera adapted primers. Libraries were generated using unique barcodes and NGS sequencing was performed using an Illumina MiSeq instrument with 2×300 paired end reads. The sequencing data was analyzed using CRISPResso2. Rates of ABE8e target adenosine conversion to guanine were calculated from the NGS data at the following genomic loci: (C) TRAC, (E) B2M, (G) PD1, (H) AAVS1, and (I) CISH. Further, indel rates were calculated from the NGS data at AAVS1 (N). (RT) refers to the population of 4 retargeting gRNAs for the AAVS1 locus. Gene loci are abbreviated as follows: (T) TRAC, (B) B2M, (P) PD1, and (C) CISH.

FIG. 14 shows that the base editor ABE8e produces fewer indels that Cas9. T cells were electroporated with mRNA encoding Cas9, Cas9 nickase (nCas9), or ABE8e and chemically modified sgRNAs targeting the AAVS1 locus. gDNA was collected at day 5, and PCR was performed to amplify the locus of interest. Sanger sequencing of this amplicon was performed and was analyzed using the TIDE INDEL program. The results comprise data from 3 healthy T cell donors.

FIG. 15 shows a flow cytometry analysis of knockout and transgene expression in primary human T cells via the base editor retargeting method. The expression of (A) TRAC (T), (B) B2M (B), (C) PD1 (P), (D) GFP, and (E) CD33-CAR was measured 7 days after gene editing using ABE8e followed by transduction with a donor template encoding either GFP or CAR via ssAAV6. The CISH (C) KO not shown. The data points are from two healthy human donors. (RT) refers to the population of 4 gRNAs needed for retargeting at AAVS1.

FIG. 16 shows that CAR-T cells generated using the base editor retargeting method exhibit effective cytokine production and can kill antigen-positive target tumor cell lines within co-cultures. (A) Schematics depicting functional assay designs and incubation times. (i) Engineered T cells are co-cultured with target cancers cells and the ability of the CAR construct to recognize a target antigen expressed by the target cancer cells is examined via detection of intracellular inflammatory cytokine production by flow cytometry. (ii) Luciferase positive target cancer cells (or control cancer cells that lack the CAR target antigen) are co-cultured with engineered T cells. Luciferase expression is assessed at 24 hours and a reduction in target cell signal indicates dying/killing of the cell population. (B) Flow cytometry analysis of intracellular IFNγ staining on CD8+ T cells post 12-hour incubation with the indicated target cells. (Note: Raji cells lack the CAR target antigen.) When the engineered T cells were co-cultured with cancer cells that express the CAR target antigen, they were activated and produced inflammatory cytokines. Importantly, matched T cells from the same donor that lack the knocked in CAR are not activated by these same cells. This demonstrates that the knocked in CAR allows the engineered T cells to specifically target CAR target antigen positive tumor cells when normally they would not. (C) Killing assay readouts at 24 hours with engineered CAR T cells cocultured at the indicated effector:target (E:T) ratio with CAR target antigen negative Raji cells. There is little to no change in bioluminescence following coculture with the CAR T cells, indicating that the CAR T cells do not kill the Raji cells. This was expected because Raji cells do not express the CAR-antigen. (D) Killing assay readouts at 24 hours with engineered CAR T cells cocultured at the indicated E:T ratio with CAR target antigen positive HL-60 cells. The results show dose dependent, tumor cell-specific killing of the HL-60 cells. (E) Killing assay readouts at 24 hours with engineered CAR T cells cocultured at the indicated E:T ratio with CAR target antigen positive MOLM-13 cells. The results show dose dependent, tumor cell-specific killing of the MOLM-13 cells.

FIG. 17 shows the base editor retargeting method is functional and efficient in other cell types. Flow cytometry analysis of CD133-CAR transgene expression was performed in edited natural killer (NK) cells. The engineered NK cells showed levels of transgene expression that are comparable to those achieved with traditional Streptococcus pyogenes Cas9 (SpCas9), which generates DSBs.

### DETAILED DESCRIPTION

All publications, including but not limited to patents and patent applications, cited in this specification are herein incorporated by reference as though set forth in their entirety in the present application.

The methods and compositions described herein are based, at least in part, on the inventors' development of protocols for multiplex genome engineering that utilize a single base editor enzyme to simultaneously knock in and knock out multiple target genes.

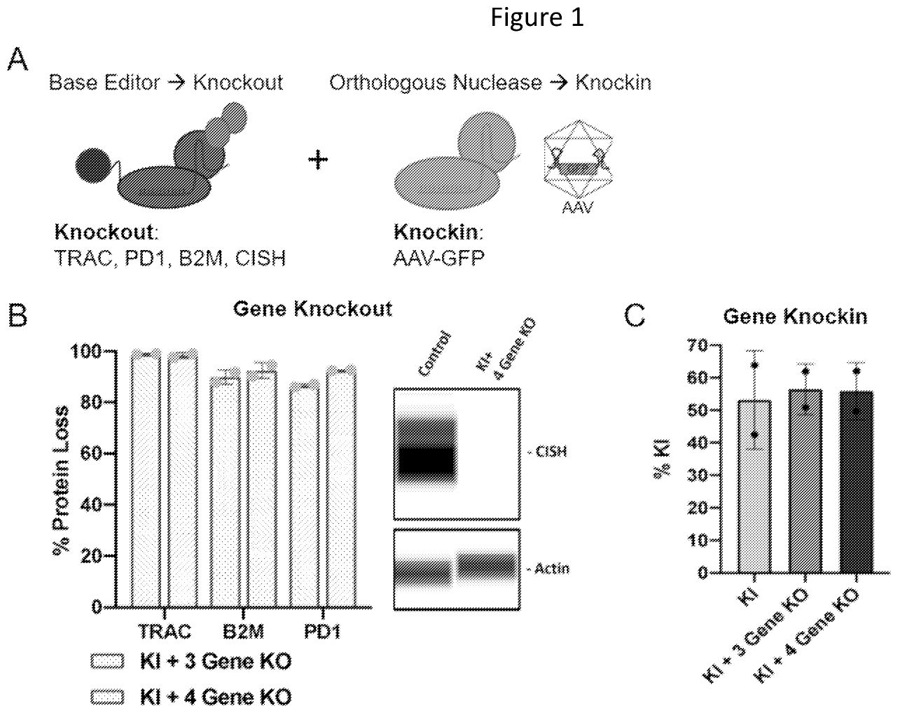

Engineered cell-based therapies, such as chimeric antigen receptor T cells, often requires multiple gene knockouts as well as gene additions. However, current knockin practices rely on random integration of viral vectors, which poses safety risks and limits efficacy. To address this shortcoming, the present inventors have developed genome engineering methods that utilize CRISPR/Cas base editors to perform targeted gene knockins and knockouts with high efficiency. While simultaneous knockins/knockouts can also be achieved using a multi-enzyme system (e.g., using a base editor for knockouts and an orthologous nuclease for knockins, as depicted in FIG. 1), the complexity of such systems is not ideal for GMP manufacturing, for which systems that require few reagents are advantageous. Thus, the inventors have developed and optimized methods that allow multiplex editing to be performed using a single enzyme. Rather than using targeted nucleases, which generate blunt-end cuts or very short overhangs that are prone to generating genotoxic chromosomal rearrangements, the inventors use base editor enzymes in their methods. Because base editors generate DNA nicks with massive overhangs, use of these enzymes allows the DNA to remain relatively stable until an HDR event occurs. As a result, the inventors' methods are safer and more controllable than the methods of the prior art. The inventors' methods are referred to herein as a “dual-nicking” methods because they use nickase enzymes to cut one strand of DNA at two adjacent sites, thereby generating a staggered double-stranded DNA break.

As is described in the Examples, the inventors discovered that the rates of transgene knockin that can be achieved using a base editor are far lower than those achieved with a Cas nuclease or Cas nickase. In an attempt to improve the efficiency of base editor-mediated knockins, the inventors designed retargeting gRNAs, which target the sequence that has been edited by the base editor. Surprisingly, they found that adding these retargeting gRNAs to the reaction improved the knockin efficiency by more than two-fold. Thus, in a first embodiment, the present invention provides methods in which a base editor is used in combination with retargeting gRNAs (referred to herein as “base editor retargeting methods”). In addition, the inventors discovered that using a base editor in the absence of retargeting gRNAs resulted in far lower (i.e., undetectable) rates of indel formation at the transgene knockin site. This was surprising, as this method was expected to induce indel formation at approximately the same rate as the Cas9 nickase-based methods of the prior art. Thus, in a second embodiment, the present invention provides methods in which a base editor is used in the absence of retargeting gRNAs (referred to herein as “basic base editor methods”).

### Methods for Producing Genetically Modified Cells:

In a first aspect, the present invention provides methods for producing genetically modified cells. In a first embodiment, the methods comprise introducing into the cells (i) a plasmid, mRNA, or protein encoding a base editor; (ii) two primary gRNAs that are complementary to the 5′ and 3′ ends of a knockin target site prior to editing; (iii) two retargeting gRNAs that are complementary to the 5′ and 3′ ends of a knockin target site after they have been edited by complexes comprising the base editor and the primary gRNAs; and (iv) a DNA donor template. This method generates cells with a targeted knockin via homology directed repair (HDR) from the DNA donor template. The methods of this first embodiment are referred to herein as “base editor retargeting methods”.

In a second embodiment, the methods comprise introducing into the cells: (i) a plasmid, mRNA, or protein encoding a base editor; (ii) at least two primary gRNAs that are complementary to the 5′ and 3′ ends of a knockin target site prior to editing; and (iii) a DNA donor template. Like the base editor retargeting method, this method generates cells with a targeted knockin via DR from the DNA donor template. However, this method is distinguished from the base editor retargeting method in that (a) it does not utilize retargeting gRNAs, and (b) the rate of indel formation that it induces at the knockin target site is less than 10%, specifically less than 1%. This methods of this second embodiment are referred to herein as “basic base editor methods”.

“Base editors” are fusion proteins that comprise a Cas nickase domain or catalytically dead Cas protein fused to a deaminase. As in CRISPR-based gene editing, base editors are targeted to a specific gene sequences using a guide RNA (gRNA). However, unlike Cas nuclease-based methods, base editing does not generate double-strand DNA breaks, making it a safer alternative. Instead, base editing uses the deaminase enzyme to modify a single base without altering the bases around it. There are two classes of base editors: cytosine base editors (CBEs) and adenine base editors (ABEs). CBEs comprise a cytidine deaminase that converts cytidine to uridine within a small editing window near the protospacer adjacent motif (PAM) site. Uridine is subsequently converted to thymidine through base excision repair, creating a cytosine (C) to thymine (T) change (i.e., a guanosine to adenine change on the opposite strand). In contrast, ABEs comprise an adenine deaminase, which creates an adenine (A) to guanosine (G) change. When a CBE is utilized, to prevent cells from repairing the modified base and encourage the cell to use the edited strand as a template for mismatch repair, a uracil DNA glycosylase inhibitor (UGI) is used to block base excision repair. In some embodiments, a UGI domain is included as part of the base editor fusion protein. In other embodiments, the UGI domain is provided to the cell as a separate component. Researchers have developed third and fourth generation base editors with improved efficiency. For example, the third generation CBE base editor BE3 (i.e., base editor 3) uses a Cas9 nickase to nick the unmodified DNA strand so that it appears to be newly synthesized to the cell, forcing the cell to repair the DNA using the deaminated strand as a template, whereas fourth generation base editor systems (i.e., base editor 4 (BE4)) employ two copies of base excision repair inhibitor UGI. In some embodiments, a BE3 or BE4 cytosine base editor is used in the methods of the present invention. In other embodiments, a CBE comprising a different deaminase, such as hA3A-BE4, hA3G-BE4, evoFERNY-BE4, or evoCDA-BE4, is used. In other embodiments, an ABE base editor, such as ABE6.3, ABE7.10, ABE8e, or ABE8.20 is used. In some embodiments, the base editor enzymes are mutated or modified to confer a desired functionality such as reduced guide-independent off-target editing, reduced guide-dependent off-target editing, an altered editing window, an altered editing context preference, an altered target site specificity, or more precise target editing.

The base editors may also be derived from other enzymatic variants. In certain embodiments, Cas9 variants with expanded PAM recognition are employed. Suitable Cas9 variants with expanded PAM recognition include, but are not limited to, SaCas9-KKH, SpCas9-VQR, SpCas9-VRER, SpCas9-NG, SpRY-Cas9, SpCas9-NRCH, and xCas9. In certain embodiments, different Cas9 orthologs or paralogs are used, such as SaCas9, FnCas9, NmeCas9, AsCas12a, FnCas12a.

As used herein, the term “knockin target site” refers to the site within the genome of the cell in which the DNA donor sequence is to be integrated. In some cases, the donor sequence is integrated at an endogenous safe-harbor locus such as the C-C Motif Chemokine Receptor 5 (CCR5), Adeno-Associated Virus Integration Site 1 (AAVS1), ROSAβgeo26 (Rosa26), albumin (ALB), or Hypoxanthine Phosphoribosyltransferase 1 (HPRT). In some embodiments, the DNA donor sequence is integrated into a transcriptionally active locus, which may be selected to confer a desired expression profile. Suitable transcriptionally active loci include, for example, T Cell Receptor Alpha Constant (TRAC), T Cell Receptor Beta Constant 1 and 2 (TRBC1 and TRBC2), CD3 Epsilon subunit (CD3E), CD19, CD16A, CD16B, NKG2A, PDCD1, NR4A1, NR4A2, NR4A3, HBG1, and HBG2. Importantly, for Cas9 to successfully bind to or cleave a target DNA sequence, a protospacer adjacent motif (PAM) must be located immediately downstream from the target site. Examples of PAM sequences are known (see. e.g., Shah et al., RNA Biology 10 (5): 891-899, 2013).

In the present methods, knockins are achieved using two gRNAs that target the base editor to the 5′ and 3′ ends of the knockin target site, at which the base editor generates single-stranded nicks that induce the cell to perform homology directed repair (ideally from the DNA donor template). “Homology directed repair (HDR)” is a cellular DNA repair pathway that can utilize either an endogenous (i.e., the sister chromosome) or exogenous piece of homologous DNA as a template to repair a patch of DNA.

A guide RNA (gRNA) comprises a sequence of at least 10 contiguous nucleotides (often 17-23 contiguous nucleotides) that is partially or wholly complementary to a target site in the genome of an organism. In some embodiments, the gRNA can be from 20 to 120 bases in length, or more. In certain embodiments, a guide molecule can be from 20 to 60 bases in length, or 20 to 50 bases, or 30 to 50 bases, or 39 to 46 bases in length. gRNAs can be designed using an online tool or an R-based program that identifies gRNAs that target a particular genomic site. The gRNAs used with the present invention may include modifications that alter their characteristics. For example, the gRNAs may be chemically modified to comprise 2′-O-methyl phosphorthioate modifications to increase their stability.

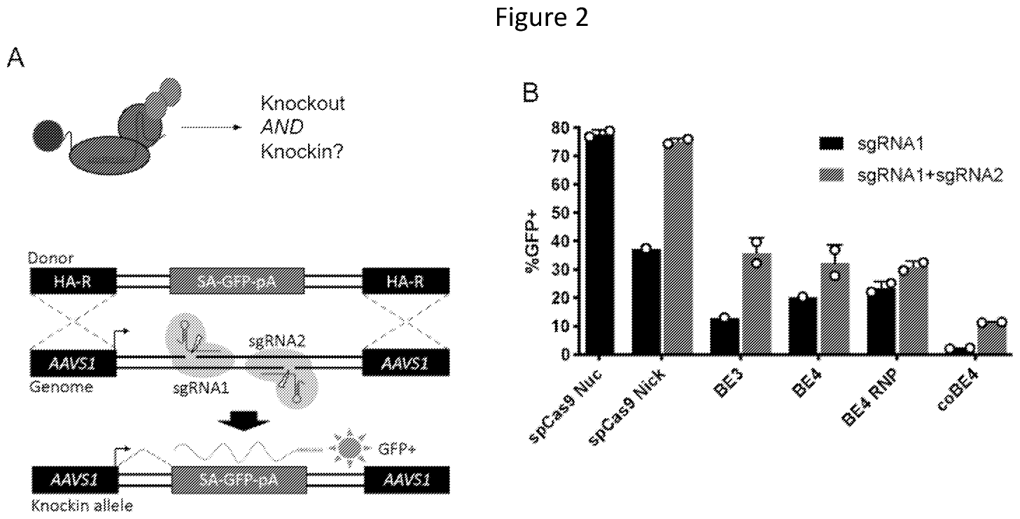

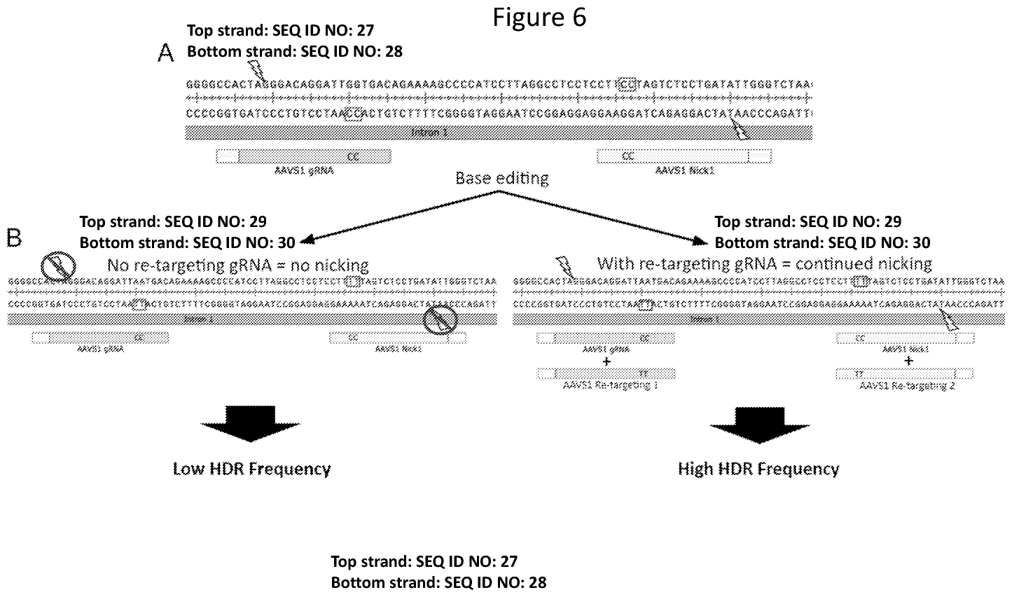

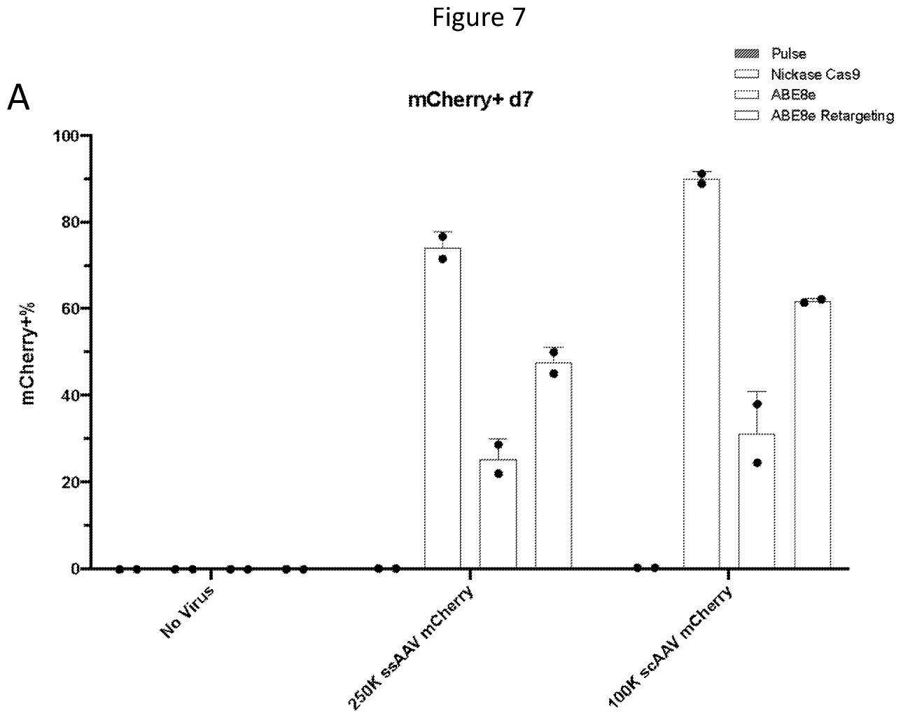

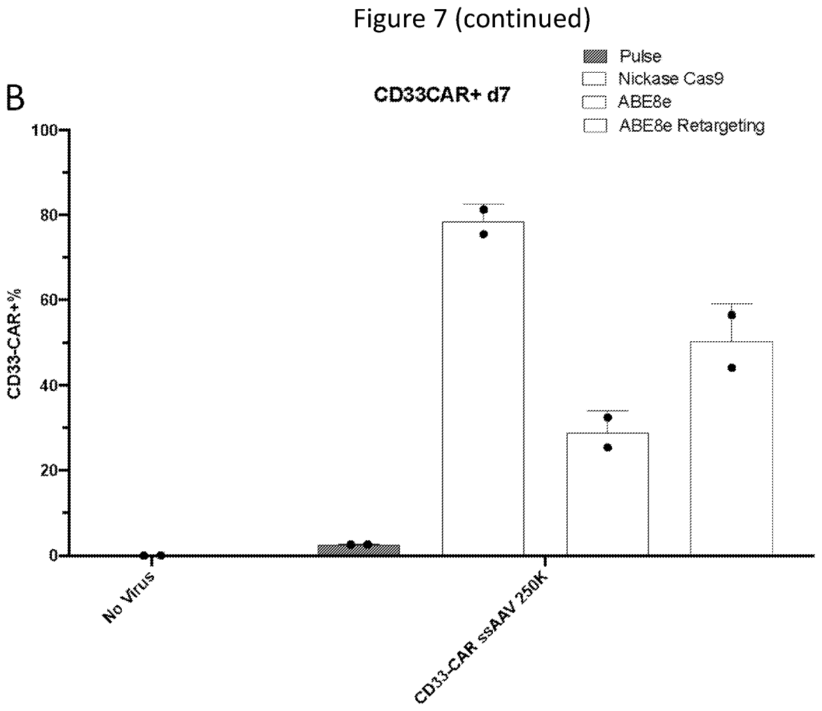

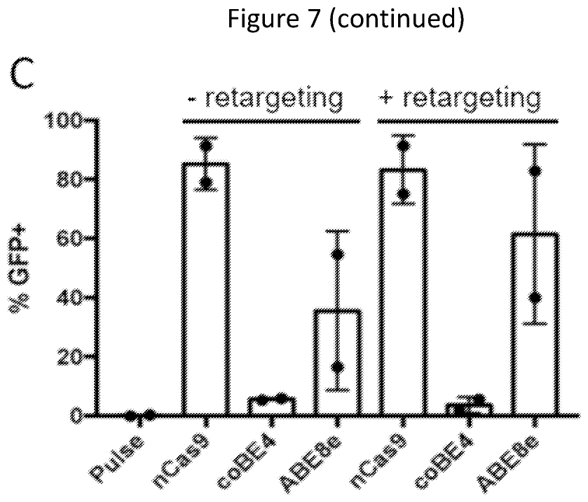

The present invention utilizes two types of gRNAs, which are referred to herein as “primary gRNAs” and “retargeting gRNAs”. The inventors discovered that when the methods of the present invention are performed using only primary gRNAs, which are complementary to the 5′ and 3′ ends of the knockin target site before it has been edited (i.e. the “wild-type” sequence), the knockin efficiency is much lower than that achieved with Cas nuclease or Cas nickase (see FIG. 2B). Thus, the inventors designed retargeting gRNAs that are complementary to the 5′ and 3′ ends of a knockin target site after they have been edited by complexes comprising the base editor and the primary gRNAs. These retargeting gRNAs ensure that the knockin target site is subjected to continuous re-nicking (see the Brief Description of FIG. 6 for a more detailed description of this process). Surprisingly, the inventors discovered that including the retargeting gRNAs in the base editing reaction increased the knockin efficiency by over two-fold (see FIG. 7).

As used herein, the terms “DNA donor template,” “donor template,” “donor sequence,” and “donor” refer to a DNA sequence that may be used by a cell as a repair template for homology-directed repair (HDR). The DNA donor template may be double-stranded DNA or single-stranded DNA (e.g. an ssODN). In certain embodiments, the DNA donor template may be less than about 100 nucleotides in length, about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, or greater than about 1000 nucleotides in length. The portion of the DNA donor template that is to be inserted into the genome of a cell may be of any length (e.g., as short as a single nucleotide or greater than ten kilobases). The inserted portion of the donor may comprise an exogenous sequence, such as a gene encoding a therapeutic protein, a non-coding RNA (e.g., an siRNA), or a selectable marker protein (e.g., a resistance gene, a fluorescent protein, a luminescent protein, a radionuclide reporter (e.g., sodium iodide symporter (NIS)), or a marker suicide gene (e.g., RQR8, truncated CD19). The inserted portion of the donor may be derived from the same species as the cell or from a different species from the cell, or it may be a chimeric sequence derived from multiple species. In the Examples, the inventors used a double-stranded DNA donor template encoding a fluorescence protein (i.e., GFP) under the control of the endogenous adeno-associated virus integration site 1 (AAVS1) promoter (SEQ ID NO: 26) to knock this gene into primary human T cells, thereby generating T cells that express GFP (FIGS. 1 and 2). Additionally, the inventors used a DNA donor template encoding a CD33-specific chimeric antigen receptor (CAR) to knock this gene into T cells, thereby generating CAR T cells (FIG. 7).

To facilitate HDR, the DNA donor template comprises “homology arms” at its 5′ end and 3′ end. “Homology arms” are DNA sequences that are complementary to regions of the genome flanking the knockin target site. In certain embodiments, the homology arms are about 20 nucleotides in length to about 1000 nucleotides in length. In certain embodiments, the homology arms are less than about 20 nucleotides in length, about 20 nucleotides in length, about 30 nucleotides in length, about 40 nucleotides in length, about 50 nucleotides in length, about 60 nucleotides in length, about 70 nucleotides in length, about 80 nucleotides in length, about 90 nucleotides in length, about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, or greater than about 1000 nucleotides in length. In some embodiments, the 5′ homology arm and the 3′ homology arm are of different lengths.

In the methods of the present invention, the base editing components (i.e., the base editor, gRNAs, and DNA donor template) may be introduced using any suitable means known in the art, including transfection, transformation, conjugation, and transduction. Other known means of delivery, such as liposomes, yeast systems, microvesicles, and gene guns are also contemplated herein. Alternatively, nucleic acids and proteins can be delivered with a pharmaceutically acceptable vehicle or encapsulated in a liposome. In some cases, cells are electroporated for uptake of the gRNAs and base editor. In some cases, a viral vector or plasmid is used to deliver the base editing components. The various base editing components may each be delivered by different means. For example, the DNA donor template may be delivered to the cell by a recombinant adeno-associated virus (rAAV; e.g., by addition of viral supernatant to culture medium) while the gRNA and base editor are encoded by plasmids that are introduced using electroporation. The base editing components may be introduced into the cells in vitro, ex vivo, or in vivo.

In certain embodiments, a viral vector is used to deliver one or more of the base editing components. As used herein, the term “viral vector” refers to a virus particle used to deliver a recombinant viral nucleic acid encoding a heterologous polypeptide into cells. The recombinant viral nucleic acid typically includes cis-acting elements for expression of the encoded heterologous polypeptide. Suitable viral vectors are known in the art and include, but are not limited to, for example, an adenovirus vector; an adeno-associated virus vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector. In preferred embodiments, the viral vector is an adeno-associated viral (AAV) vector.

In some embodiments, the viral vector is an AAV vector with an alternative natural or engineered serotype, such as AAV1, AAV2, AAV3, AAV4, AAV5, AAV6, AAV7, AAV8, or AAV9.

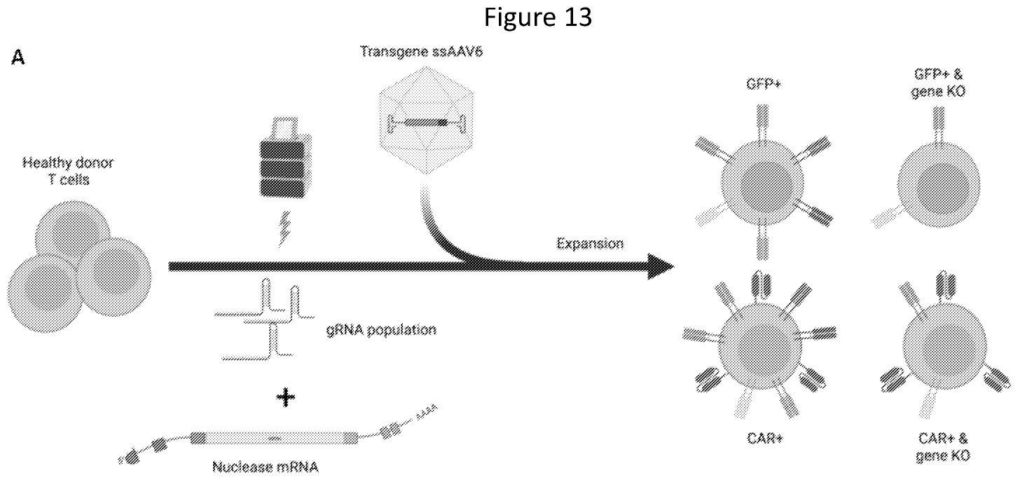

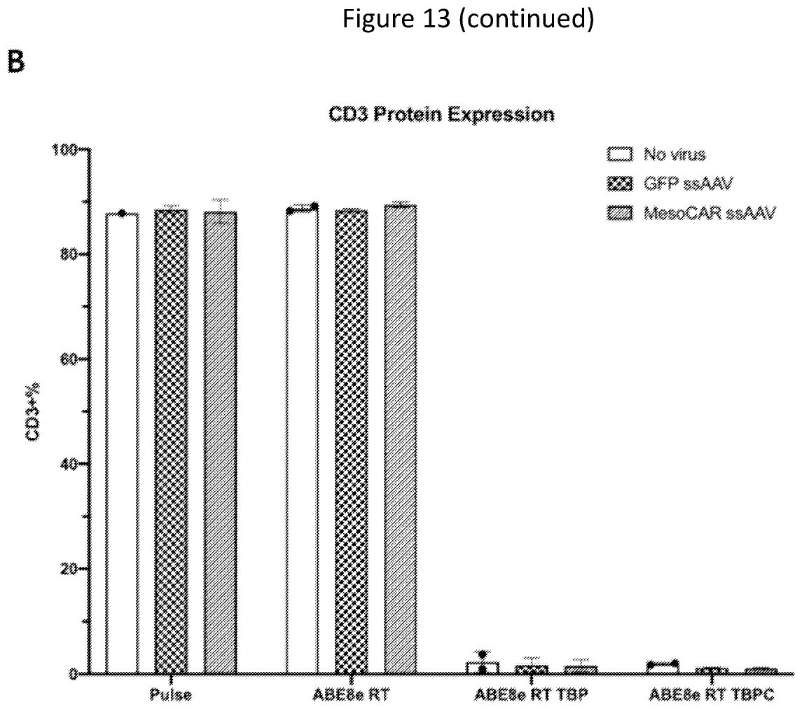

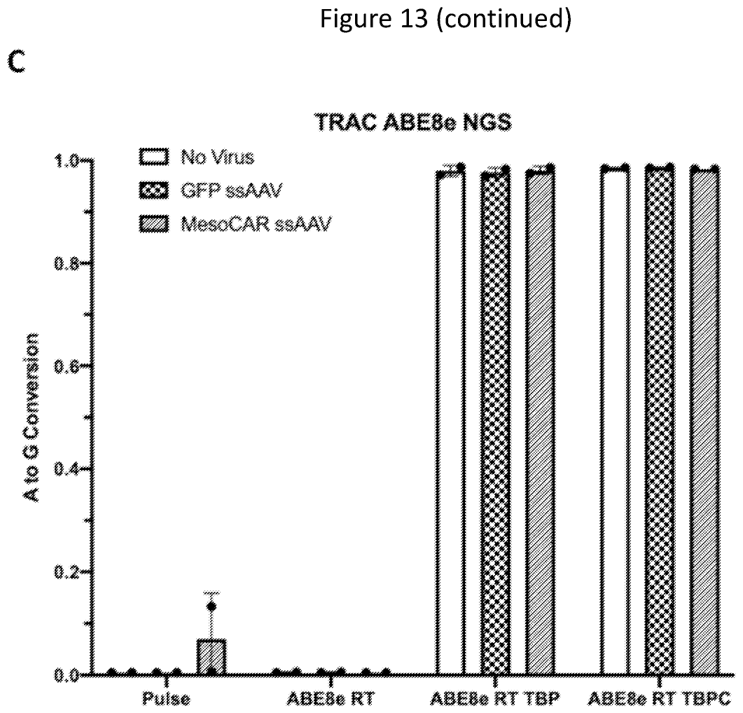

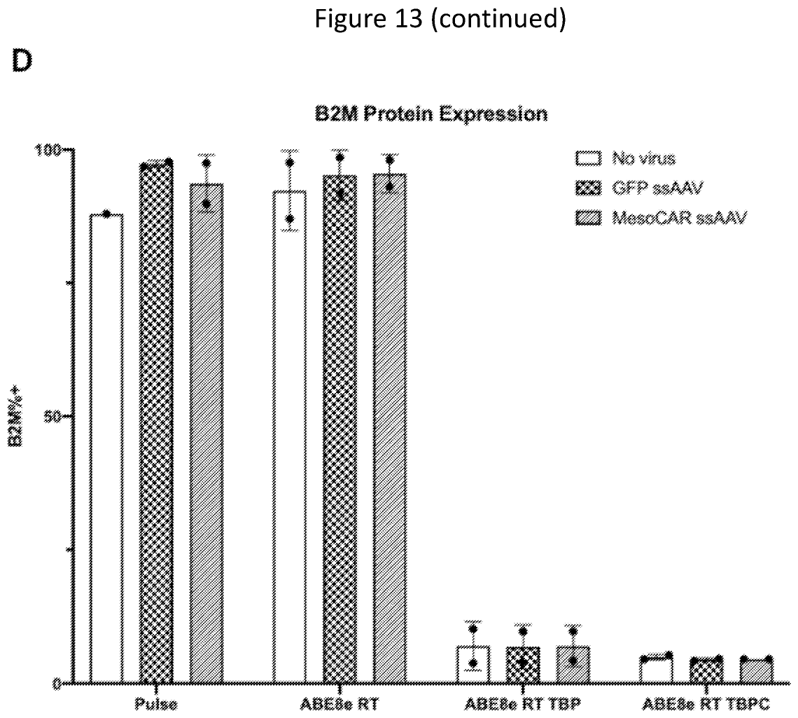

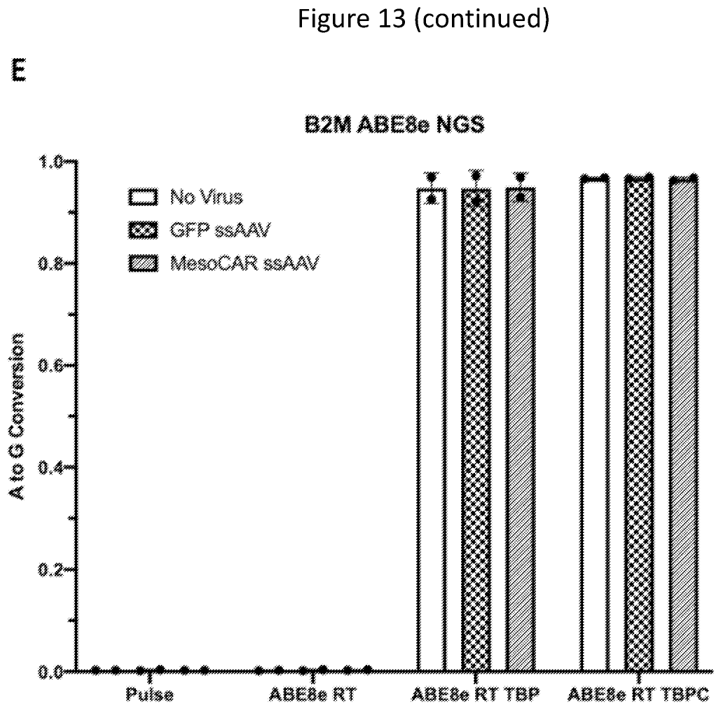

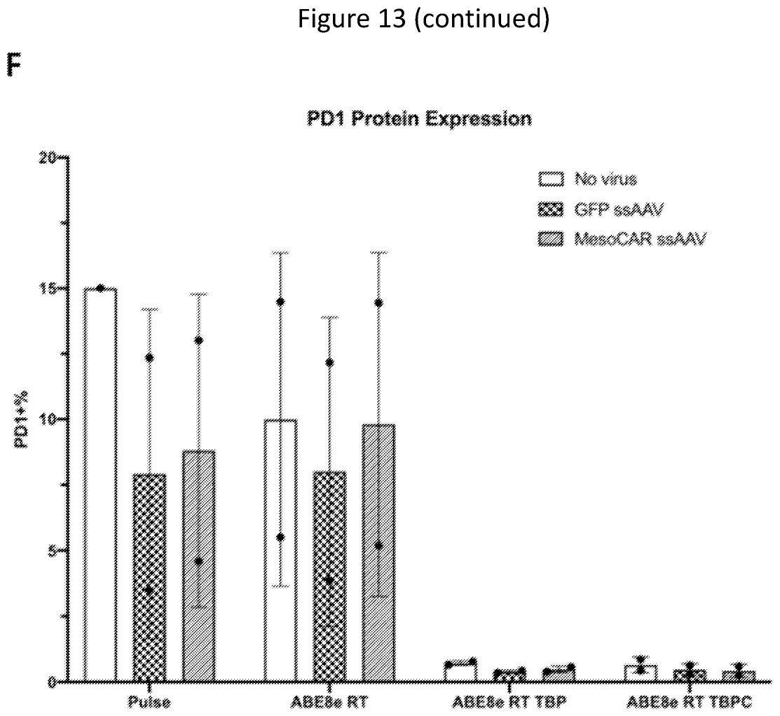

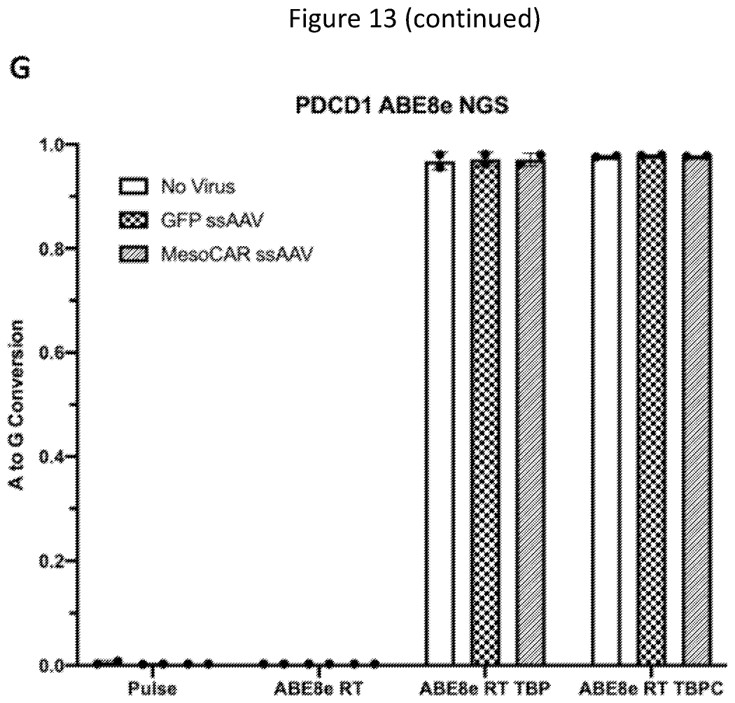

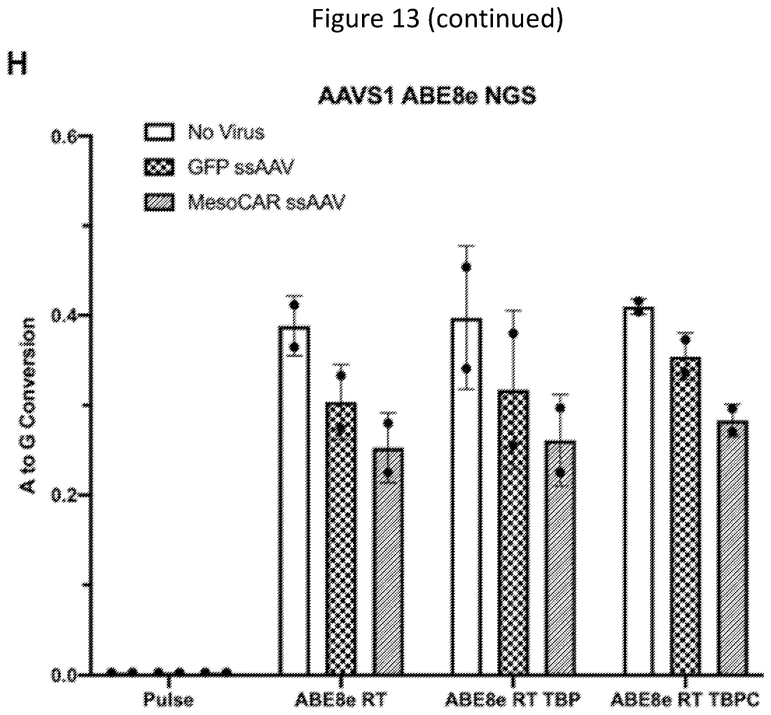

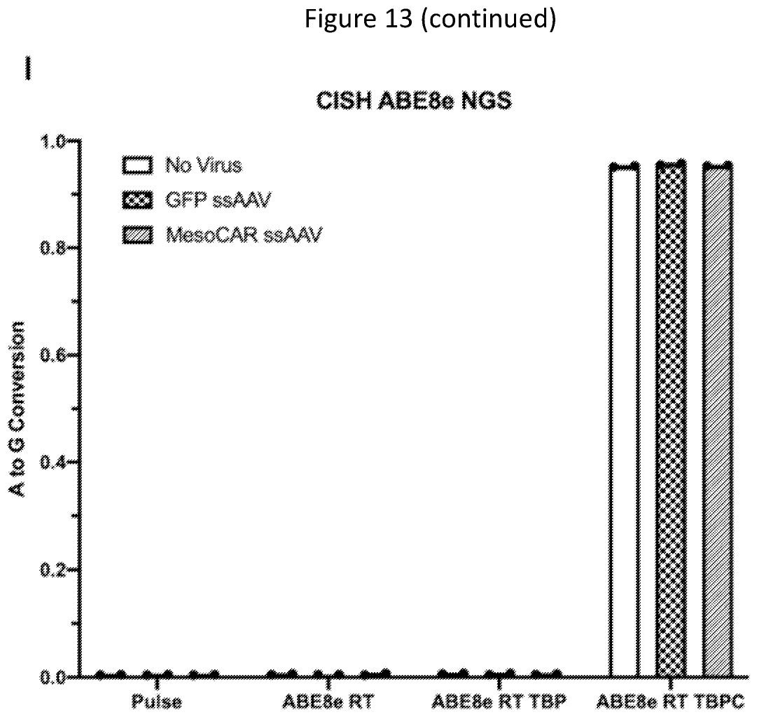

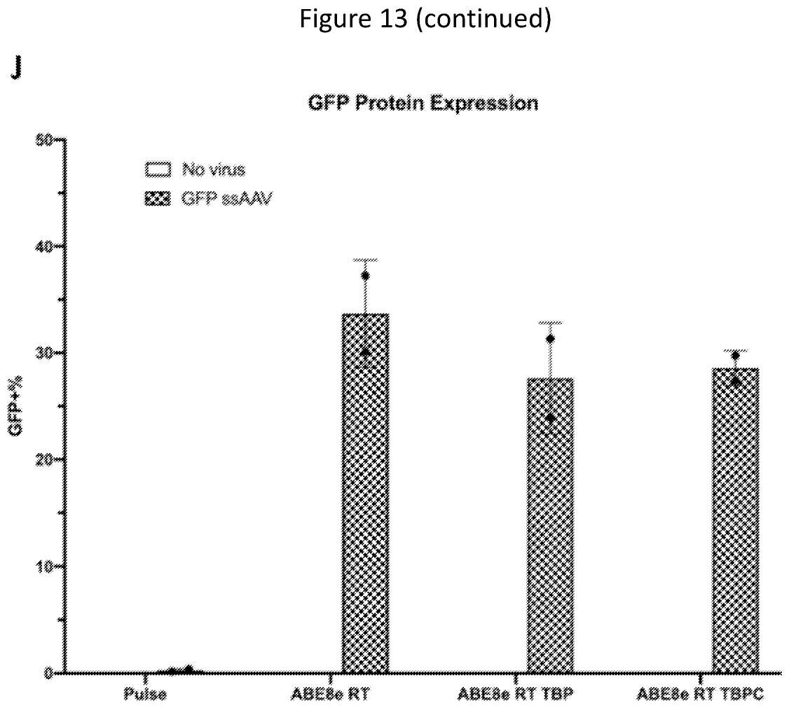

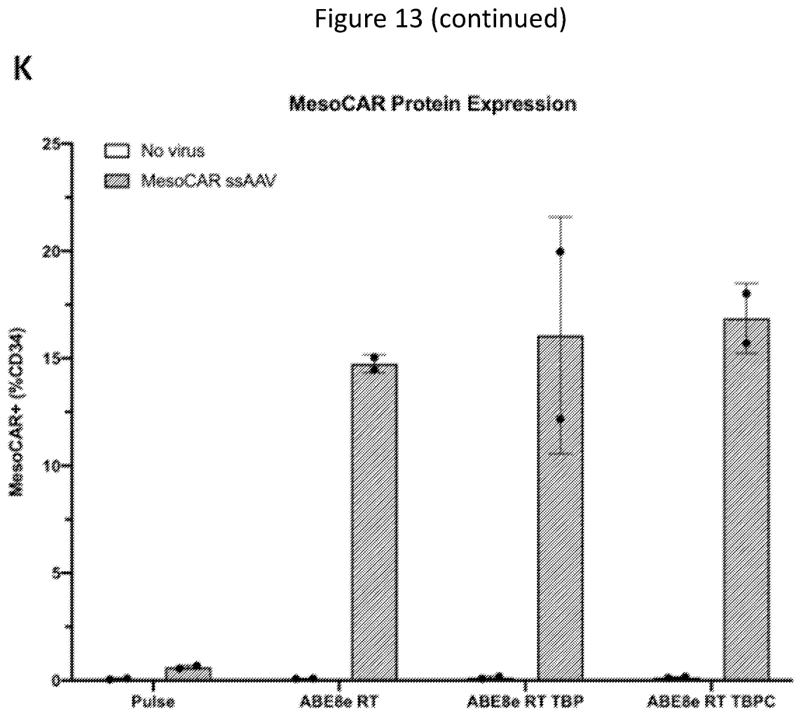

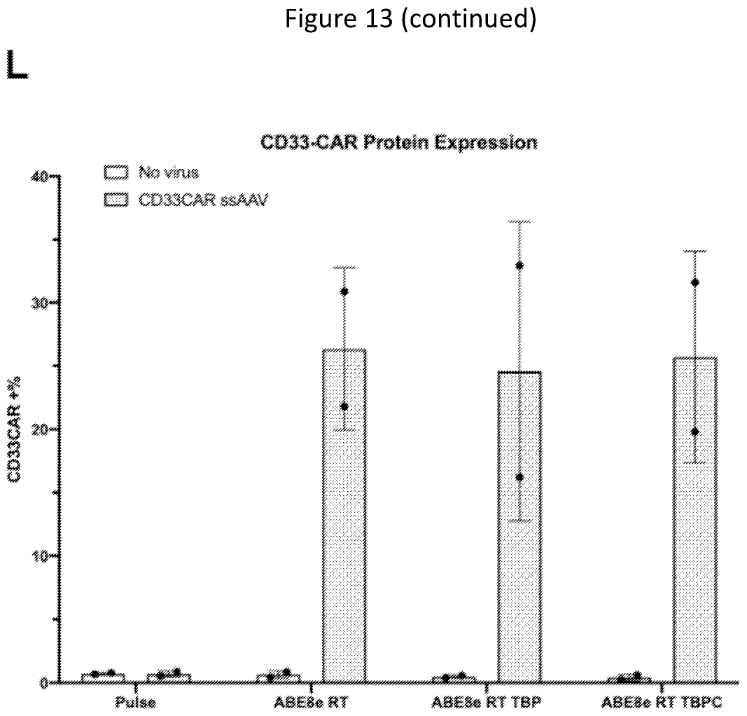

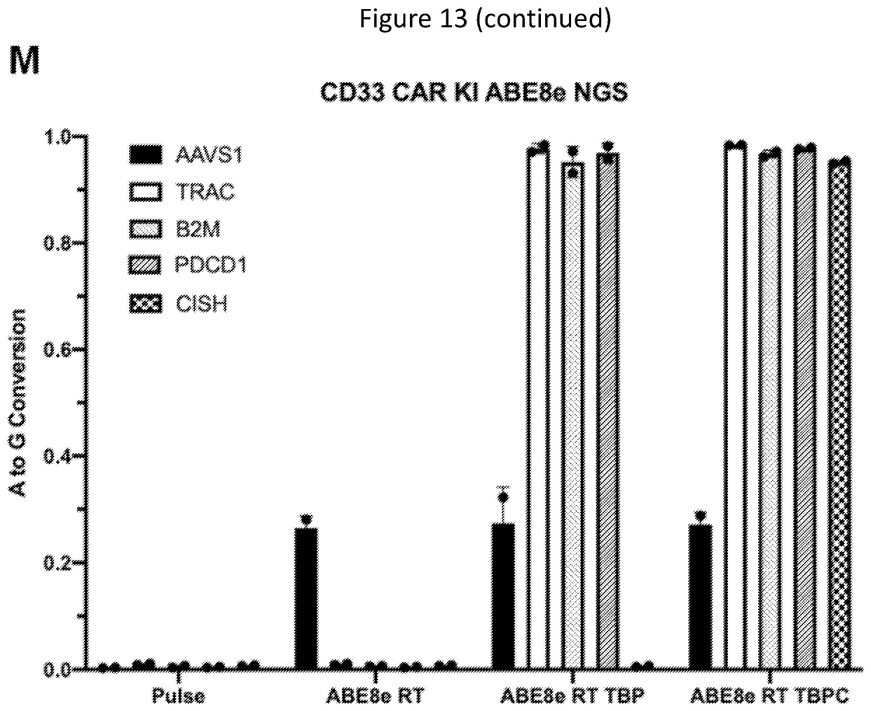

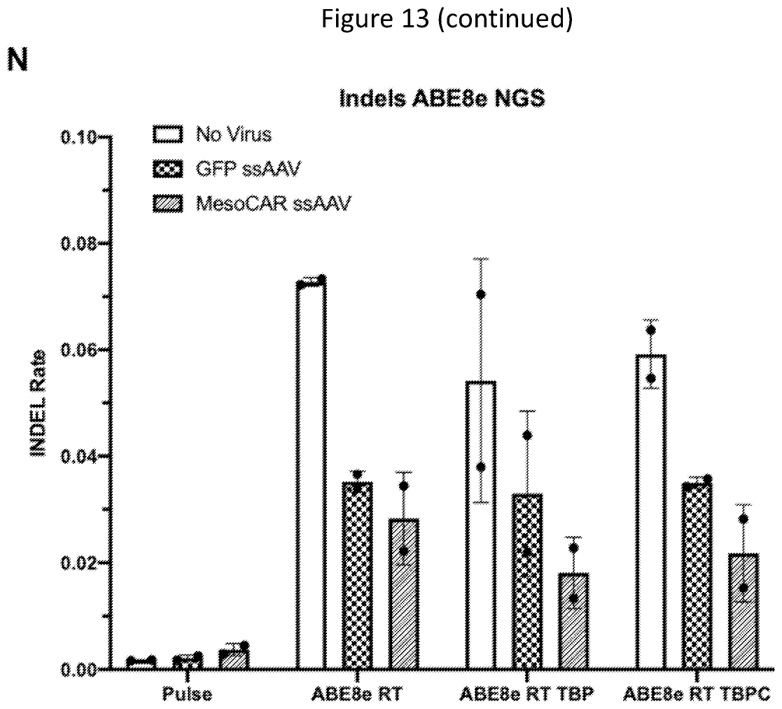

The inventors have demonstrated that the methods of the present invention can be used to simultaneously generate gene knockins and gene knockouts. The methods may be used to simultaneously introduce/modify at least one, at least two, at least three, at least four, at least five, at least six, at least seven, at least eight, at least nine, or at least 10 genes. In the Examples, the inventors demonstrate that their methods can be used to simultaneously knock in one gene (i.e., CD33 CAR) and knock out 4 genes (i.e., TRAC, B2M, PD1, and CISH; see, e.g., FIG. 13). Thus, in some embodiments, the method produces genetically modified cells in which at least five genes have been modified (i.e., knocked out or introduced via knock in).

Knockouts may be generated using any suitable method known in the art. For example, a target gene may be knocked out via disruption of a start codon or introduction of a premature stop codon. In these embodiments, it may only be necessary to modify a single base in the target gene, and only one gRNA may be necessary. In these embodiments, the methods may further comprise introducing into the cells: one or more primary gRNAs that are complementary to a knockout target site prior to editing. Alternatively, a target gene may be knocked out via disruption of intron/exon splice sites. In these embodiments, the methods may further comprise introducing into the cells: at least one gRNA that targets a slice donor and at least one gRNA that targets a splice acceptor within the knockout target site. As used herein, the term “knockout target site” refers to a site within the genome of the cell that is mutated to make a gene inoperative.

In some embodiments, the methods of the present invention are used to knock in multiple genes. In these embodiments, the methods further comprise introducing into the cells: at least one additional set of reagents that target a different knockin target site to generate cells with two or more targeted knockins. For the base editor retargeting methods, each additional set of gRNAs comprises: (i) two primary gRNAs that are complementary to two knockin target sites prior to editing; (ii) two retargeting gRNAs that are complementary to the two knockin target sites after they have been edited by complexes comprising the base editor and the primary gRNAs; and (iii) a DNA donor template. For the basic base editor methods, each additional set of gRNAs comprises: (i) two primary gRNAs that are complementary to two knockin target sites prior to editing; and (ii) a DNA donor template.

The inventors have demonstrated that the inclusion of retargeting gRNAs in their base editing methods significantly increases the knockin efficiency (i.e., frequency at which the desired transgene knockin occurs) that can be achieved. While integration rates of over 20% were achieved using the basic base editor method (see FIG. 2), integration rates ranging from 40-65% were achieved using the base editor retargeting method (see FIG. 7). The knockin efficiency of a base editing method will depend on several factors, including the knockin target site and the size of the DNA donor template construct utilized. The knockin efficiency can be determined, for example, on the genomic level by EditR analysis of Sanger sequencing traces or by next generation sequencing (NGS), or on the protein level by flow cytometry. Thus, in some embodiments, the method generates cells with a targeted knockin with at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% efficiency.

Further, the inventors also demonstrated that the rate of indel formation at the knockin target site produced by the basic base editor method was substantially lower than the rate produced by the base editor retargeting method. An “indel” is a short polymorphism created by the insertion or deletion (indel) of a small number of bases in a DNA sequence. As used herein, the term “rate of indel formation” refers to the frequency at which an indel is generated at a particular genomic site as a consequence of the base editing method. Specifically, the inventors demonstrated that that indel formation was less than 20%, preferably less than 10%, and suitably undetectable using the basic base editor method. This extremely low rate of indel formation may prove to be critical for avoiding toxic DSBs in applications involving primary human cells. The indel formation rate can be determined, for example, by TIDE INDEL analysis of sequencing data. Thus, in some embodiments, the rate of indel formation is less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or is undetectable (i.e., below the limit of detection of a particular detection method).

The methods of the present invention may be performed using any eukaryotic cell. In some embodiments, the cells are lymphohematopoietic cells. As used herein, the term “lymphohematopoietic cells” refers to T cells, natural killer (NK) cells, B cells, CD34+ hematopoietic stem progenitor cells (HSPCs), and other cells involved in the production of lymphocytes and cells of blood, bone marrow, spleen, lymph nodes, and thymus. In some embodiments, the cells are T cells or NK cells. In other embodiments, the cells are pluripotent stem cells or progenitor cells capable of differentiating into T cells or NK cells. In some embodiments, the cells are freshly isolated primary cells or cells obtained from a frozen aliquot of a primary cell culture. In some embodiments, the cells are mammalian cells. In particular embodiments, the cells are human cells. In some embodiments, the cell is an engineered cell that contains an exogenous, recombinant, synthetic, and/or otherwise modified polynucleotide. In certain embodiments, the cells are derived from a human patient in need of treatment for a disease or condition.

In some embodiments, the methods are applied to T cells. Methods of electroporating T cells are known in the art. For example, the electroporation conditions used for T cells can comprise 1400 volts, pulse width of 10 milliseconds, 3 pulses. In some embodiments, the Amaxa® Nucleofector® is used to perform electroporation with protocol EO-100 or EO-115. Following electroporation, T cells may be allowed to recover in a cell culture medium. In some cases, electroporated T cells are allowed to recover in the cell culture medium for about 5 to about 30 minutes (e.g., about 5, 10, 15, 20, 25, 30 minutes). Preferably, the recovery cell culture medium is free of antibiotics and other selection agents. The recovered T cells may then be subjected to an expansion protocol, e.g., they may be cultured in a T cell expansion medium. In some cases, the T cell expansion medium is complete CTS OpTmizer T-cell Expansion medium.

The inventors have designed the present methods to allow one to simultaneously knock in and knock out genes to generate cells that can be used as therapeutics. For example, the inventors used their base editing methods to simultaneously knock in a gene encoding a chimeric antigen receptor (CAR; i.e., a CD33-specific CAR) and knock out several T cells genes (i.e., TRAC, PDCD1, B2M, and CISH) that make these cells immunogenic. Such knockouts can be used, for example, to generate cells for use as allogenic anti-tumor cell therapeutics or for the treatment of autoimmune diseases. In the Examples, the inventors demonstrate that their base editor retargeting method can be used to knock out four genes using 8 independent gRNAs with over 80% efficiency, preferably more than 90% efficiency (see FIG. 13). Thus, in some embodiments, the methods generate cells with a targeted knockout with at least 90% efficiency.

In some embodiments, the DNA donor template encodes a CAR, such that the methods are be used to generate genetically modified cells that express a CAR. As used herein, the term “chimeric antigen receptor (CAR)” refers to an artificially constructed fusion protein comprising an extracellular antigen-binding domain of an antibody (e.g., a single chain variable fragment (scFv)) operably linked to a transmembrane domain and at least one intracellular domain. Generally, the antigen-binding domain of a CAR has specificity for a particular antigen expressed on the surface of a target cell of interest. For example, T cells can be engineered to express a CAR that specifically binds to the molecule CD19, which is expressed on B-cell lymphoma. Thus, in some embodiments, the CAR comprises a CAR antigen-binding domain that specifically binds to a tumor antigen. A “tumor antigen” is an antigen that is preferentially expressed on the surface of a tumor cell and not expressed on normal, healthy cells. To generate allogenic anti-tumor cell therapeutics that are not limited by donor-matching, cells can be engineered to both knock in a nucleic acid encoding a CAR and knock out genes responsible for donor matching (e.g., TCR and HLA markers).

In some embodiments, the DNA donor template encodes a T cell receptor (TCR). In specific embodiments, the TCR specifically binds to a tumor antigen. In some embodiments, the DNA donor template encodes both a TCR and a CAR, and the TCR and CAR bind to different antigens.

The gRNAs used with the present invention may be used to target any genomic site for gene knockin or knockout. In the Examples, the inventors used gRNAs that target the AAVS1 locus to knock a gene into this locus in T cells (see Table 1). Thus, in some embodiments, the one or more gRNAs are selected from the sequences set forth in Table 1 (i.e., SEQ ID NOs: 1-6 and 33-38). Additionally, the inventors used gRNAs to knock out various T cell genes (see Table 2). Thus, in some embodiments, the one or more gRNAs include one or more sequences set forth in Table 2 (i.e., SEQ ID NOs: 7-25). Additional gRNA sequences that can be used with the present invention can be found in the inventors' prior work described in PCT Application No. PCT/US2019/022049, which is hereby incorporated by reference in its entirety regarding the entire disclosure.

TABLE 1
gRNAs for gene knockins at the AAVS1 locus

| Gene | ERNA name | S′-gRNA Sequence-3′ |
|---|---|---|
| AAVS1 | Nick 1 | GTCACCAATCCTGTCCCTAG (SEQ ID NO: 1) |
| AAVS1 | Nick 1 ABE retargeting | GTCGCCGGTCCTGTCCCTAG (SEQ ID NO: 2) |
| AAVS1 | Nick I CBE retargeting | GTTATTAATCCTGTCCCTAG (SEQ ID NO: 3) |
| AAVS1 | Nick 2 | CTTCCTAGTCTCCTGATATT (SEQ ID NO: 4) |
| AAVS1 | Nick 2 ABE retargeting | CTTCCTGGTCTCCTGATATT (SEQ ID NO: 5) |
| AAVS1 | Nick 2 CBE retargeting | CTTTTTAGTCTCCTGATATT (SEQ ID NO: 6) |
| TRAC | TRAC EX.1 LN.1 | AGAGGATCAGGGTTAGGACA (SEQ ID NO: 33) |
| TRAC | TRAC Ex.1 LN.2 | TCAGGGTTCTGGATATCTGT (SEQ ID NO: 34) |
| TRAC | TRAC EX.1 LN.3 | GCTGGTACACGGCAGGGTCA (SEQ ID NO: 35) |
| TRAC | TRAC Ex.1 RN.1 | CCAGAACCCTGACCCTGCCG (SEQ ID NO: 36) |
| TRAC | TRAC Ex.1 RN.2 | CCTGCCGTGTACCAGCTGAG (SEQ ID NO: 37) |
| TRAC | TRAC Ex.1 RN.3 | AAGTCTGTCTGCCTATTCAC (SEQ ID NO: 38) |

TABLE 2
gRNAs for gene knockouts in T Cells

| | gRNA | | | Target | |
|---|---|---|---|---|---|
| Gene | name | 5′-gRNA Sequence-3′ | Orientation | base(s) | Predicted Outcome |
| PDCD1 | Ex. 1 SD | CACCTACCTAAGAACCAT | Antisense | C7 | Splice donor |
| | | CC (SEQ ID NO: 7) | | | disruption: GT → |
| | | | | | AT |
| PDCD1 | Ex. 2 SA | GGAGTCTGAGAGATGGA | Antisense | C6 | Splice acceptor |
| | | GAG (SEQ ID NO: 8) | | | disruption: AG → |
| | | | | | AA |
| PDCD1 | Ex. 3 SA | TTCTCTCTGGAAGGGCAC | Antisense | C7 | Splice acceptor |
| | | AA (SEQ ID NO: 9) | | | disruption: AG → |
| | | | | | AA |
| PDCD1 | Ex. 3 SD | GACGTTACCTCGTGCGGC | Antisense | C8 | Splice donor |
| | | CC (SEQ ID NO: 10) | | | disruption: GT → |
| | | | | | AT |
| PDCD1 | Ex. 4 SA | CCTGCAGAGAAACACACT | Antisense | C2 | Splice acceptor |
| | | TG (SEQ ID NO: 11) | | | disruption: AG → |
| | | | | | AA |
| PDCD1 | Ex. 2 | GGGGTTCCAGGGCCTGTC | Antisense | C7, C8 | pmSTOP induction: |
| | pmSTOP | TO (SEQ ID NO: 12) | | | TGG (Trp) → TAG, |
| | | | | | TGA, TAA |
| PDCD1 | Ex. 3 | CAGTTCCAAACCCTOGTG | Sense | C7 | pmSTOP induction: |
| | pmSTOP | GT (SEQ ID NO: 13) | | | CAA (Gln) → TAA |
| | 1 | | | | |
| PDCD1 | Ex. 3 | GGACCCAGACTAGCAGC | Antisense | C5, C6 | pmSTOP induction: |
| | pmSTOP | ACC (SEQ ID NO: 14) | | | TGG (Trp) → TAG, |
| | 2 | | | | TGA, TAA |
| TRAC | Ex. 1 SD | CTTACCTGGGCTGGGGAA | Antisense | C5 | Splice donor |
| | | GA (SEQ ID NO: 15) | | | disruption: GT → |
| | | | | | AT |
| TRAC | Ex. 3 SA | TTCGTATCTGTAAAACCA | Antisense | C8 | Splice acceptor |
| | | AG (SEQ ID NO: 16) | | | disruption: AG → |
| | | | | | AA |
| TRAC | Ex. 3 | TTTCAAAACCTGTCAGTG | Sense | C4 | pmSTOP induction: |
| | pmSTOP | AT (SEQ ID NO: 17) | | | CAA (Gln) → TAA |
| | 1 | | | | |
| TRAC | Ex. 3 | TTCAAAACCTGTCAGTGA | Sense | C3 | pmSTOP induction: |
| | pmSTOP | TT (SEQ ID NO: 18) | | | CAA (Gln) → TAA |
| | 2 | | | | |
| B2M | Ex. 1 SD | ACTCACGCTGGATAGCCT | Antisense | C6 | Splice donor |
| | | CC (SEQ ID NO: 19) | | | disruption: GT → |
| | | | | | AT |
| B2M | Ex. 3 SA | TCGATCTTATGAAAAAGAC | Antisense | C6 | Splice acceptor |
| | | AG (SEQ ID NO: 20) | | | disruption: AG → |
| | | | | | AA |
| B2M | Ex. 2 | CTTACCCCACTTAACTAT | Antisense | C7, C8 | pmSTOP induction: |
| | pmSTOP | CT (SEQ ID NO: 21) | | | TGG (Tr) → TAG, |
| | | | | | TGA, TAA |
| CD3E | Ex.2 SD | ACTCACCTGATAAGAGGC | | | |
| | | AG (SEQ ID NO: 22) | | | |
| PDCD1 | EXISD | CACCTACCTAAGAACCAT | | | |
| | | CC (SEQ ID NO: 23) | | | |
| B2M | Ex.ISD | CTTACCCCACTTAACTAT | | | |
| | | CT (SEQ ID NO: 24) | | | |
| CISH | Ex.2.SD | CTCACCAGATTCCCGAAG | | | |
| | | GT (SEQ ID NO: 25) | | | |

In some embodiments, the methods are used to produce cells that have been genetically modified to be “universally acceptable” for therapeutic applications. In a preferred embodiment, the cells are genetically engineered T cells that are suitable as universally acceptable T cells for therapeutic applications. As used herein, the term “universally acceptable” is used to describe cell products that can be administrated to a patient without the use of cross-matching or immunosuppression. Such cells can be obtained, for example, by disrupting the expression of alloreactive proteins (i.e., proteins that generate a T cell response) or immunomodulatory proteins (i.e., proteins that activate or suppress the immune system). For example, the inventors have engineered T cells in which several alloreactive or immunomodulatory genes (i.e., TRAC, PDCD1 (programmed cell death 1), B2M (beta-2 microglobulin), and CISH (cytokine-inducible SH2-containing protein)) have been knocked out (see FIG. 1). Alloreactive proteins in T cells refers to proteins on the surface of the T cell that recognize peptide-allogeneic-MHC complexes that were not encountered during thymic development and manifests itself clinically as transplant rejection and graft-versus-host disease (GVHD). Thus, in some embodiments, method is used to generate T cells with a targeted knockout of a gene selected from TRAC, PDCD1, B2M, and CISH.

### Genetically Modified Cells and Uses Thereof:

In a second aspect, the present invention provides genetically modified cells obtained according to the methods disclosed herein. These cells may be useful for many applications including, without limitation, studies of cell biology and gene function, disease modeling, and cell-based therapeutics, such as gene therapies and cancer immunotherapies. For example, in some embodiments, the genetically modified cells of the present invention are used in a treatment for cancer.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

### Definitions

So that the compositions and methods provided herein may more readily be understood, certain terms are defined.

Gene “knockin (KI)” is a process in which an exogenous gene is inserted at a specific locus in a genome. The term “targeted knockin” refers to the exogenous gene after it has been inserted.

Gene “knockout (KO)” is a process in which an endogenous gene is mutated to make it inoperative. The term “targeted knockout” refers to the inoperative gene after it has been mutated.

As used herein the term “cancer” refers to an abnormal mass of tissue in which the growth of the mass surpasses and is not coordinated with the growth of normal tissue. A cancer or tumor can be defined as “benign” or “malignant” depending on: the degree of cellular differentiation, rate of growth, local invasion, and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule.

As used herein, the terms “engineered” and “synthetic” are used interchangeably to refer to something that has been manipulated by the hand of man.

The terms “genetically modified” and “genetically engineered” are used interchangeably herein and refer to a prokaryotic or eukaryotic cell that includes an exogenous polynucleotide, regardless of the method used for insertion. Genetic engineering can be performed at the DNA, RNA, or epigenetic level. Genetic modifications include: (i) deletion of an endogenous gene (i.e., knockout); (ii) introduction of a recombinant nucleic acid encoding a wild-type or mutant form of an endogenous or exogenous protein (i.e., knockin); (iii) introduction of an RNA molecule (e.g. small-interfering RNA (siRNA), short hairpin RNA (shRNA), anti-sense RNA, and micro RNA (miRNA)) that interferes with the functional expression of a protein; or (iv) altering the promoter or enhancer elements (i.e., regulatory elements) of one or more endogenous genes. It is understood that item (ii) includes replacement of an endogenous gene (e.g., by homologous recombination) with a gene encoding an altered or entirely different protein, and that item (iv) includes modification or manipulation of the regulatory regions of a target gene or of any region that is contiguous with a target gene (e.g., up to 5 KB on either side of the target sequence). Genetic engineering also includes altering an endogenous gene to produce a protein having additions (e.g., a heterologous sequence), deletions, or substitutions (e.g., point mutations).

The terms “nucleic acid” and “nucleic acid molecule,” as used herein, refer to a compound comprising a nucleobase and an acidic moiety, e.g., a nucleoside, a nucleotide, or a polymer of nucleotides and includes the term “polynucleotide” and “oligonucleotide” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, chromosome, chromatid, or other naturally occurring nucleic acid molecule. On the other hand, a nucleic acid molecule may be a non-naturally occurring molecule, e.g., a recombinant DNA or RNA, an artificial chromosome, artificial plasmid, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term“plasmid” refers to a circular double-stranded DNA construct used as a cloning vector. A plasmid can form an extrachromosomal self-replicating genetic element in some eukaryotes or prokaryotes, or it can be integrated into the host chromosome.

The term “messenger RNA (mRNA)” refers to is a single-stranded RNA molecule that is complementary to one of the DNA strands of a gene. The mRNA is an RNA version of the gene that leaves the cell nucleus and moves to the cytoplasm to be translated into protein.

As used herein, the term “deaminase” refers to an enzyme, natural or synthetic, that catalyzes the conversion of an amine group on a nucleic acid base to a carbonyl.

The terms “protein” and “polypeptide” are used interchangeably herein to designate a series of amino acid residues connected to by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs. “Protein” and “polypeptide” are often used in reference to relatively large polypeptides, whereas the term “peptide” is often used in reference to small polypeptides, but usage of these terms in the art overlaps. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent.

As used herein, “treating” or “treatment” describes the management and care of a subject for the purpose of combating a disease, condition, or disorder. Treating includes the administration of an antibody or composition of present invention to prevent the onset of the symptoms or complications, to alleviate the symptoms or complications, or to eliminate the disease, condition, or disorder.

The “subject” may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. Further, modifying may refer to altering the chemical structure of a nucleic acid base, for example, via deamination, alkylation, methylation, cross-linking, oxidation, reduction, carboxylation, or formylation. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated.

Various exemplary embodiments of compositions and methods according to this invention are now described in the following non-limiting Examples. The Examples are offered for illustrative purposes only and are not intended to limit the scope of the present invention in any way. Indeed, various modifications in addition to those shown and described herein will become apparent to those skilled in the art from the foregoing description and the following examples and fall within the scope of the appended claims.

### EXAMPLES

### Example 1

In the following example, the inventors describe a method that produces multigene knockouts and knockins with high efficiency using DNA base editors. By utilizing base editors rather than Cas nucleases to knock-in DNA cassettes, this novel approach minimizes the risks associated with the introduction of double-strand breaks (DSBs).

In an initial experiment, the inventors performed simultaneous knockouts and knockins using a multi-enzyme system comprising a base editor and an orthogonal nuclease nickase (FIG. 1A). In these methods, the base editor and its respective gRNAs were used generate gene knockouts via base editing of critical nucleotides, while the orthogonal nickase and its respective guide were used to generate a local area of genomic instability that stimulates homology directed repair (HDR). Using this combined system, they were able to maintain high (90+%) gene knockout efficiencies while simultaneously knocking-in a transgene (with 50+% efficiency), all without generating DSBs (FIG. 1B-C).

Next, the inventors tested the ability of a simplified, single-enzyme system to perform both the gene knockouts and the gene knockins. Specifically, they utilized the deaminase function of base editors to perform gene knockouts and the Cas9 nickase function of base editors to perform gene knockins (FIG. 2A). They compared the ability of several enzymes to knockin a GFP gene from a double-stranded DNA (dsDNA) donor template into an AAVS1 integration site. In this experiment, they tested several cytosine base editors, including BE3, BE4, BE4 RNP, and coBE4. BE4 RNP is a ribonucleoprotein complex comprising BE4 and a gRNA. This complex is formed before it is applied to the cells by incubating the components together. coBE4 is a codon-optimized mRNA encoding BE4, which comprises Cas9 and APOBEC domains that are not of human origin. The results of this experiment showed that while SpCas9 nickase generates knockins with high efficiency (74.2%) using two gRNAs, all the tested cytosine base editors generate knockins with much lower efficiencies (FIG. 2B).

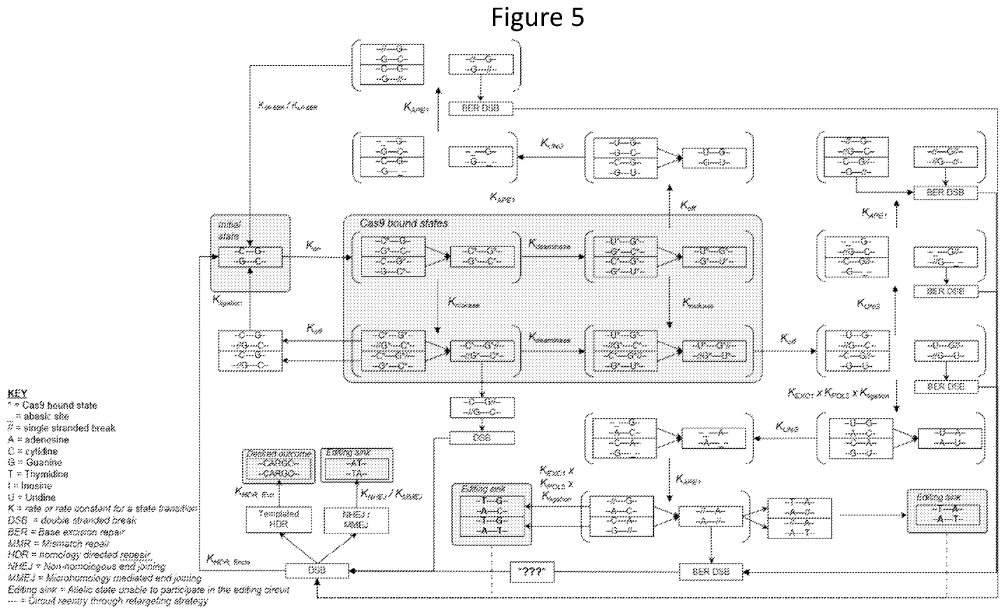

The inventors reasoned that the reduced HDR rate produced by the base editors was the result of the deaminase-mediated nucleotide conversion (i.e., the base editing reaction), as the original gRNA may no longer recognize the knockin target site once it has been edited. This results in a shortened timeframe of nicking activity and an overall decrease in HDR efficiency. Thus, to increase the knockin efficiency of the single-enzyme system, the inventors designed retargeting gRNAs that are complementary to the sequence created by deamination by the base editor, allowing nicking to continue on the edited sequence for a longer period of time (FIG. 6). The inventors tested this “base editor retargeting method” by knocking in two genes (i.e., a gene encoding GFP and a gene encoding a CD33-specific chimeric antigen receptor (CAR)) into T cells. The T cells were electroporated with mRNA encoding either Cas9 nickase or the adenine base editor ABE8e and a cocktail of dual-nicking gRNAs, with or without retargeting gRNAs. The T cells were then incubated with AAV carrying a dsDNA donor template targeting the AAVS1 locus. The results of these experiments showed that inclusion of the retargeting gRNAs increased the knockin efficiency of the single-enzyme system more than two-fold (to 60+%), such that it is comparable to that achieved with Cas9 nickase (FIG. 7A-B). However, the retargeting gRNAs did not improve the knockin efficiency of coBE4 (FIG. 7C). This suggests that a more complex mechanism may underlie CBE knockin, as is diagrammed in FIG. 5.

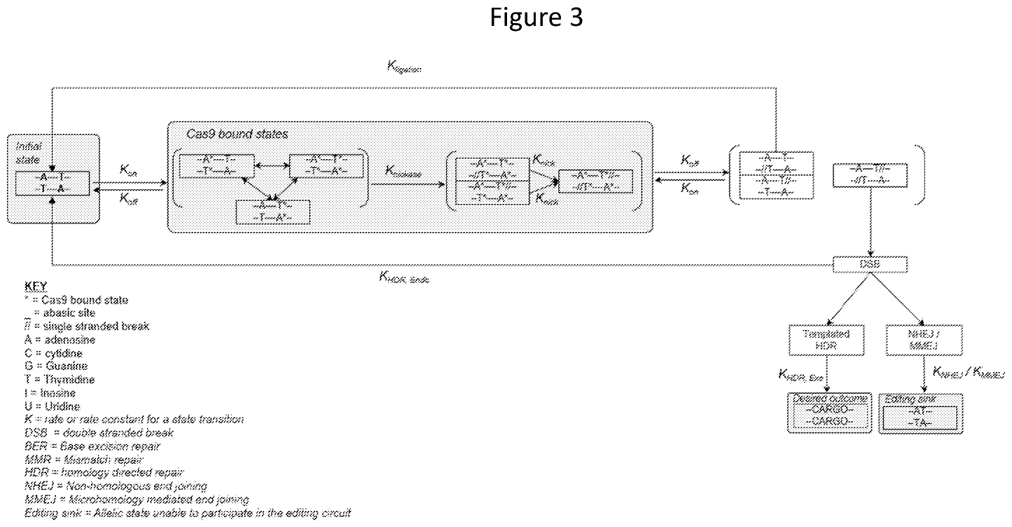

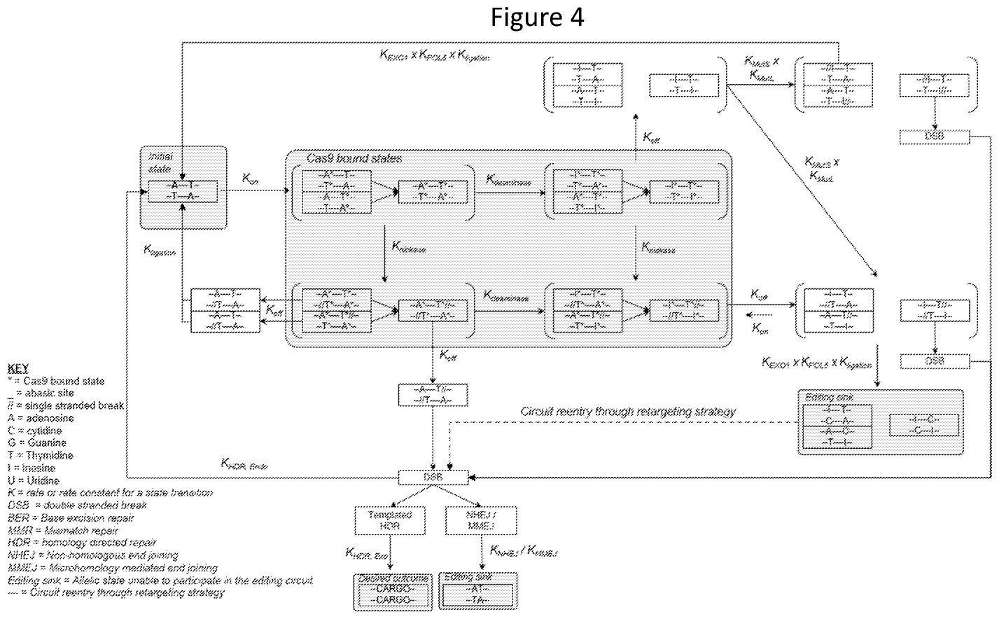

In FIGS. 3-5, the inventors describe the propose mechanisms by which various enzymes perform gene knockins in combination with two gRNAs. Specifically, FIG. 3 shows the proposed mechanism for a Cas9 nickase, FIG. 4 shows the proposed mechanism for an ABE, and FIG. 5 shows the proposed mechanism for a CBE. The inventors hypothesize that retargeting gRNAs increase the knockin efficiency at the DNA level, and they envision that the knockin rates of their base editing methods may be altered by increasing or decreasing the rates of the various transitions shown in these hypothesized editing pathways. This could be accomplished, for example, by modulating specific enzymes or other components involved in DNA repair processes (e.g., homology directed repair, mismatch repair, nucleotide excision repair, short patch base excision repair, long patch base excision repair, cross-link repair, non-homologous end joining repair, and micro-homology end joining repair). These components could be modulated, for example, using small molecule inhibitors, protein engineering, siRNA-mediated gene silencing, or gene editing.

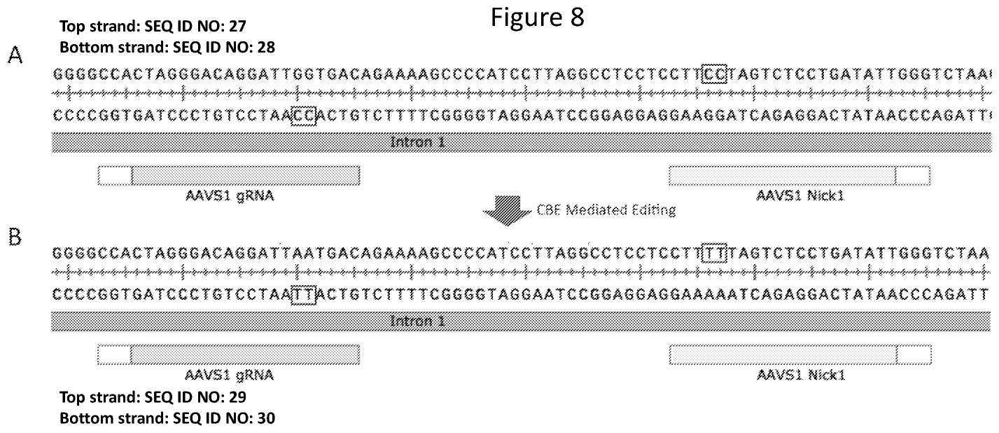

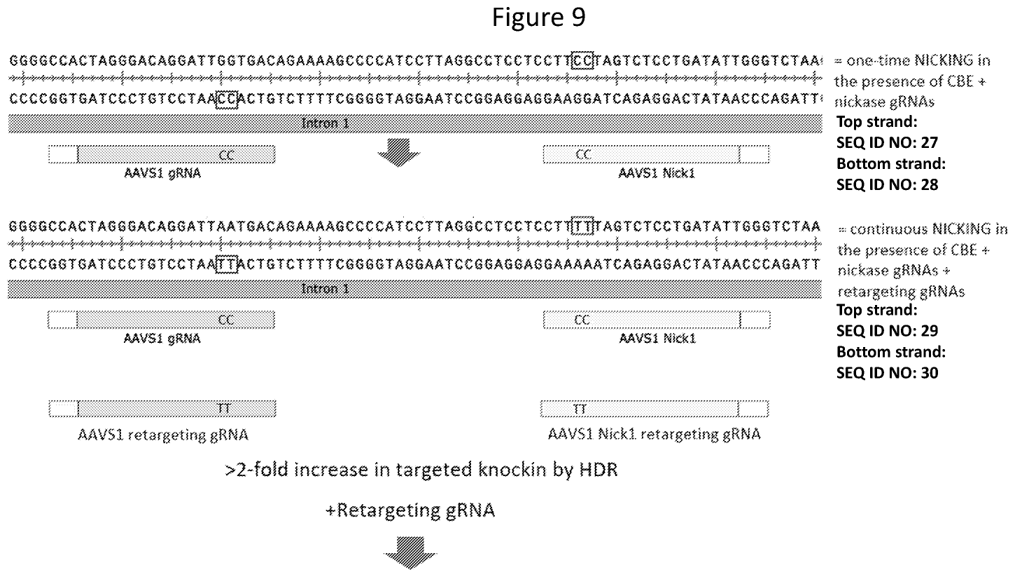

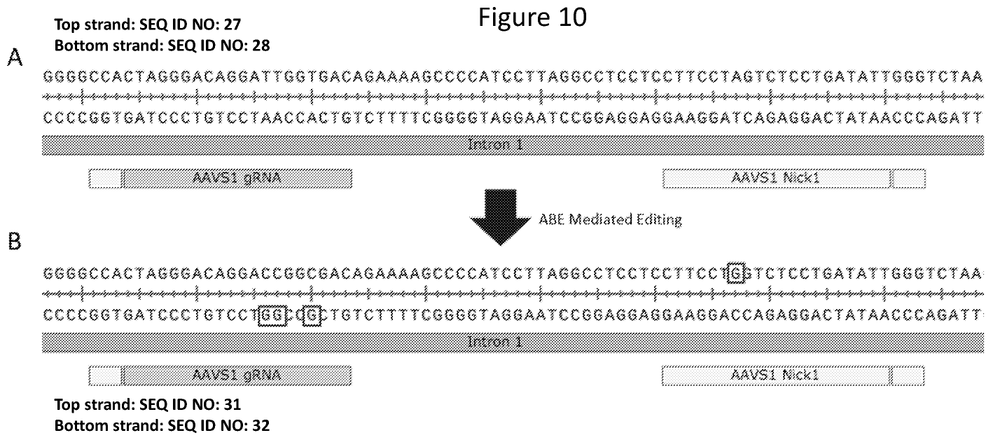

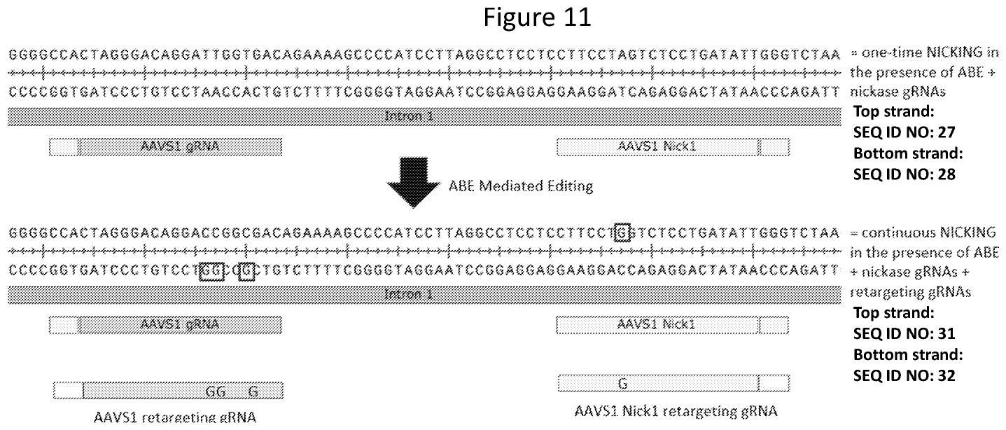

FIGS. 8-11 provide schematic depictions showing how both cytosine base editors (CBEs; FIGS. 8 and 9) and adenine base editors (ABEs; FIGS. 10 and 11) can be used in the base editor retargeting method.

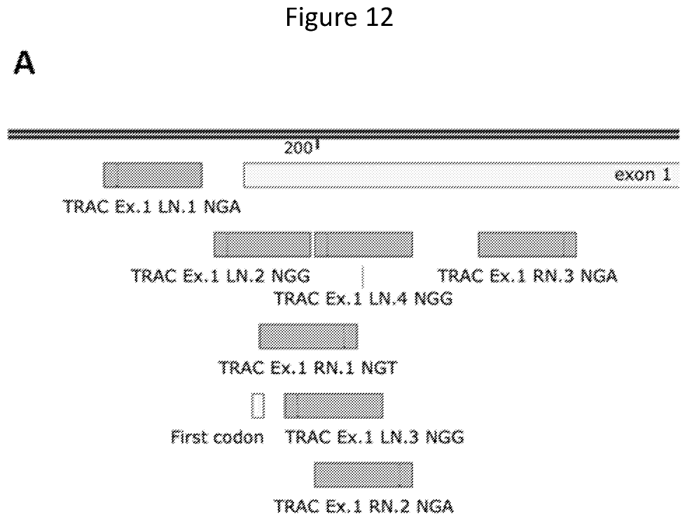

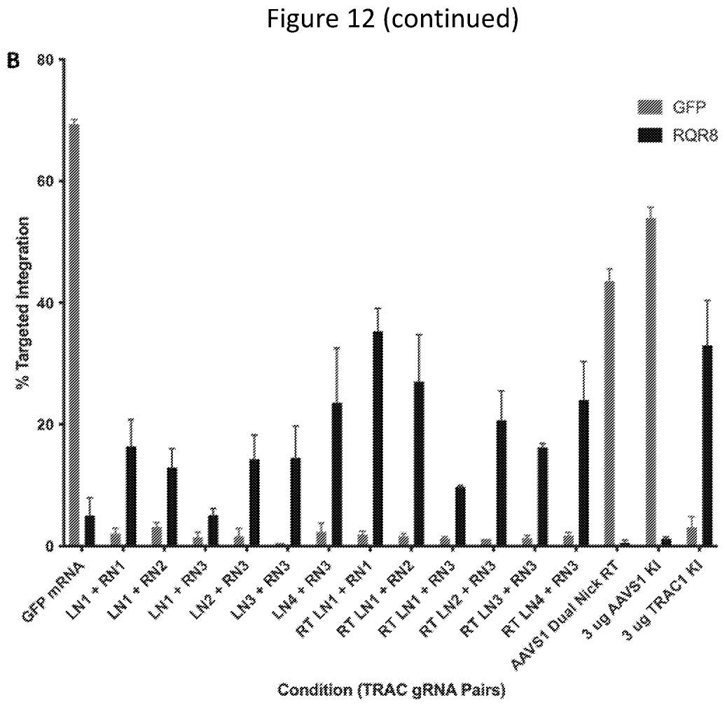

To demonstrate that the base editor retargeting method works at loci other than AAVS1, the inventors used their single-enzyme system to target the T-cell receptor α constant (TRAC) locus. In this experiment, primary human T cells were electroporated (using an Amaxa™ 4D-Nucleofector™) with mRNA encoding the adenine base editor ABE8e and a cocktail of dual-nicking gRNAs, with or without retargeting gRNAs (FIG. 12A). The T cells were then incubated with AAV6 vectors carrying a dsDNA donor template encoding GFP or CAR-T2A-RQR8 (i.e., anti-mesothelin (MSLN) CAR, CD19 CAR, and CD33 CAR) that target the TRAC locus. The results of these experiments showed that the use of the retargeting gRNAs also increased the knockin efficiency at this second target locus (FIG. 12B).

Using a standardized base editor retargeting method workflow, the inventors generated healthy and stably edited T cell populations with higher degrees of genetic engineering than can be achieved using any previously described methodology (FIG. 13). Using up to 8 independent chemically modified guides, they were able to achieve over 90% gene KO while simultaneously achieving knockin rates of over 25% with a single base editor (ABE8e). The editing rates detected via next generation sequencing (NGS) were strongly correlated with the editing rates detected using protein expression data, suggesting that the gene knockout was a direct result of the DNA changes. Also, critically, the rates of indel formation induced by the retargeting approach stay below 7% at the gene KI site, which is much lower than in previously described systems for transgene insertion that achieve efficient KI rates (see, e.g., Cell 154(6):1380-9, 2013). This is critical due to the toxicity of DSBs in primary human cells. Thus, this system strikes a previously unfound balance between the transgene KI rate and indel formation rate.

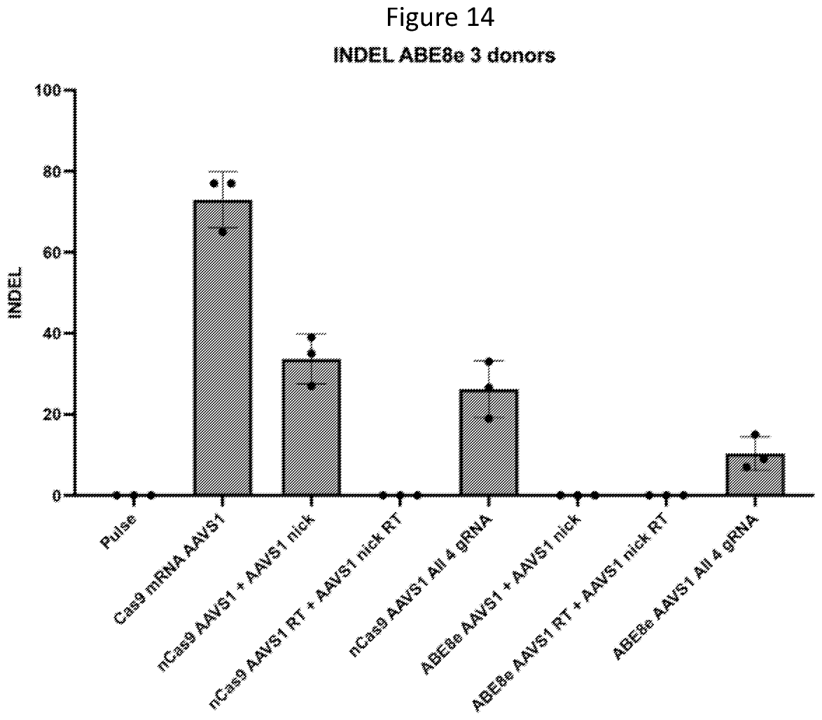

Next, the rates of indel formation generated by Cas9, Cas9 nickase, and ABE8e in the single-enzyme methods were compared (FIG. 14). Imprecise repair of double-strand breaks (DSBs) results in the introduction of random mutations, such as indels. Thus, the results of this comparison suggest that the base editor retargeting system may produce lower levels of toxic DSBs and translocations while still achieving comparable levels of gene KI to Cas9 nickase. Additionally, the results show that more basic method in which a base editor is used in the absence of retargeting gRNAs produced almost undetectable levels of indels. Lower levels of DSBs may be a critical for editing CD34+ and other stem-like cells in which DSBs are particularly toxic, even if the method results in a lower rate of gene integration.

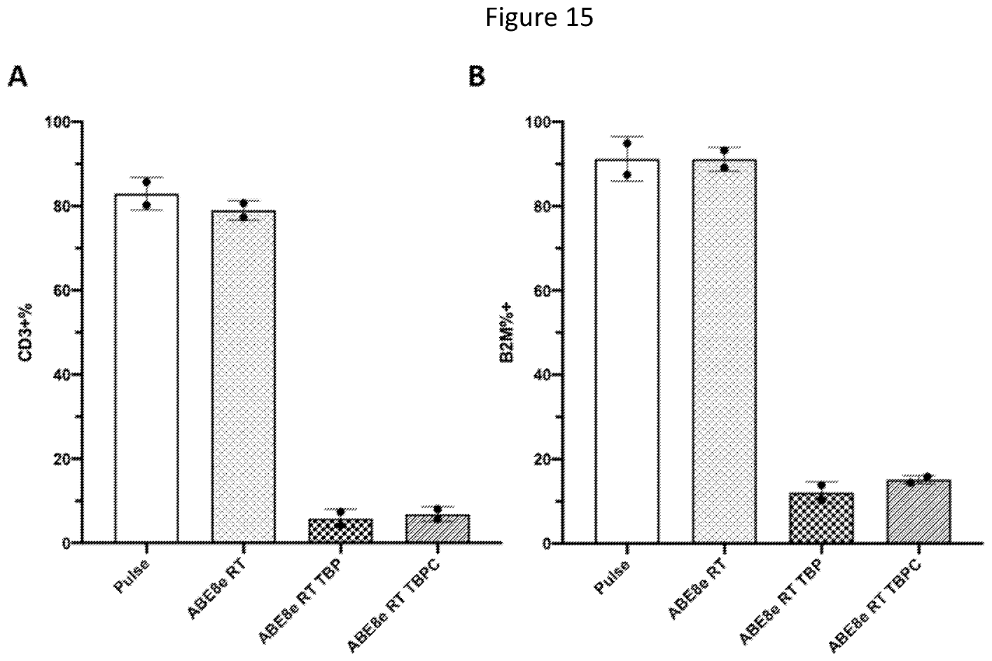

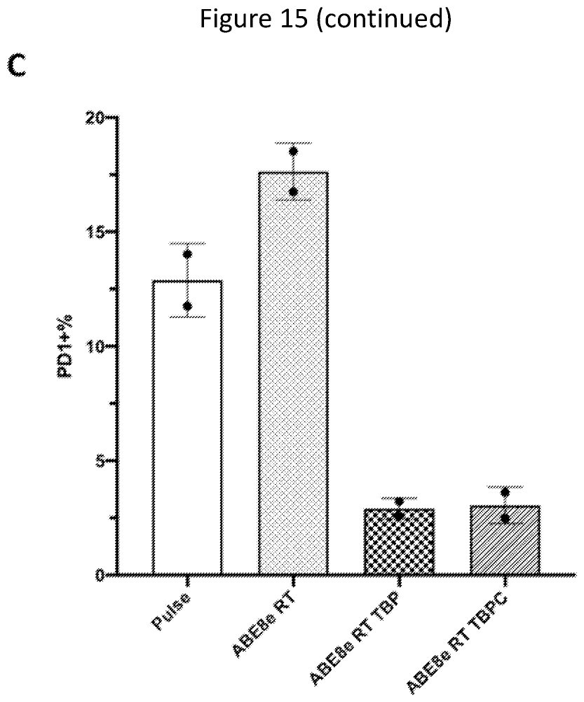

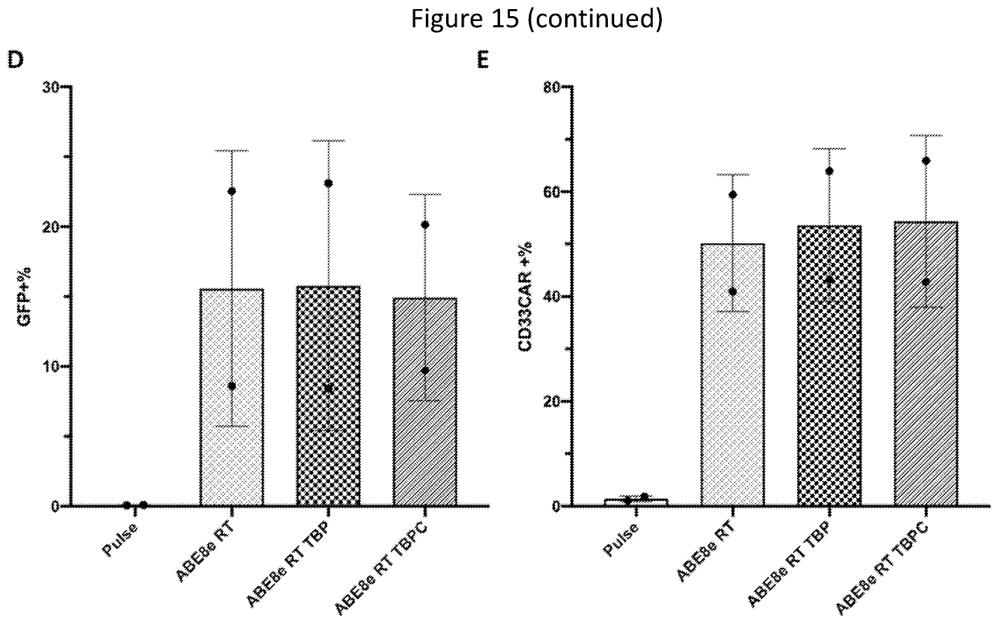

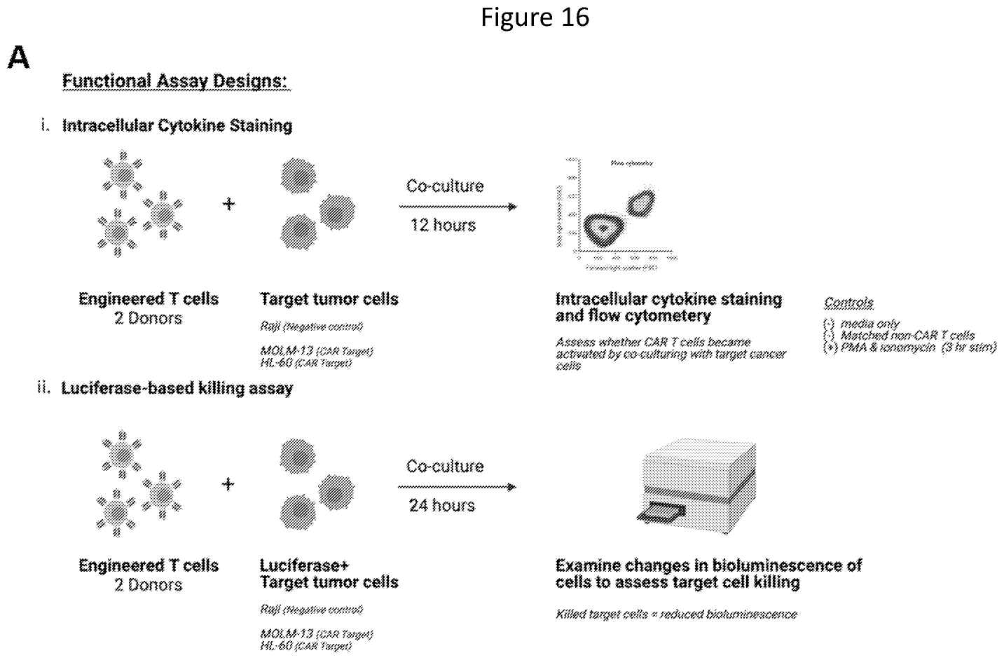

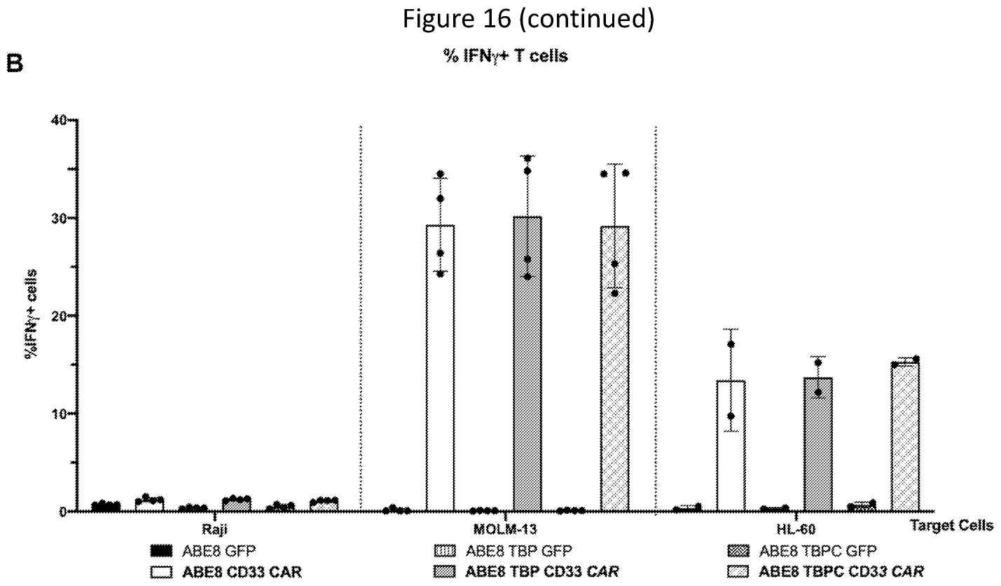

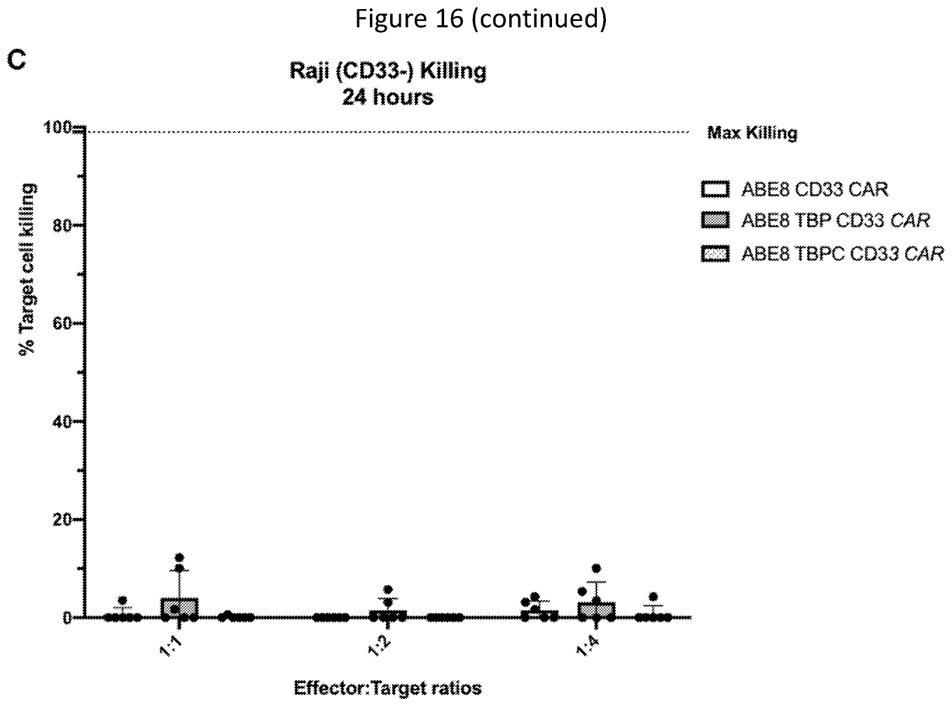

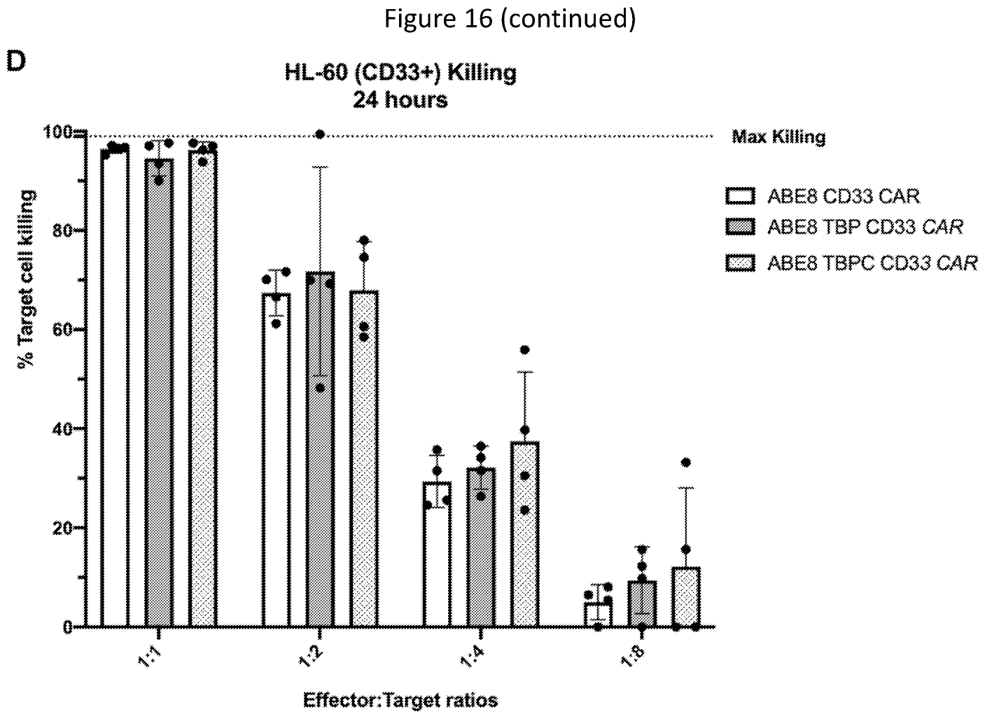

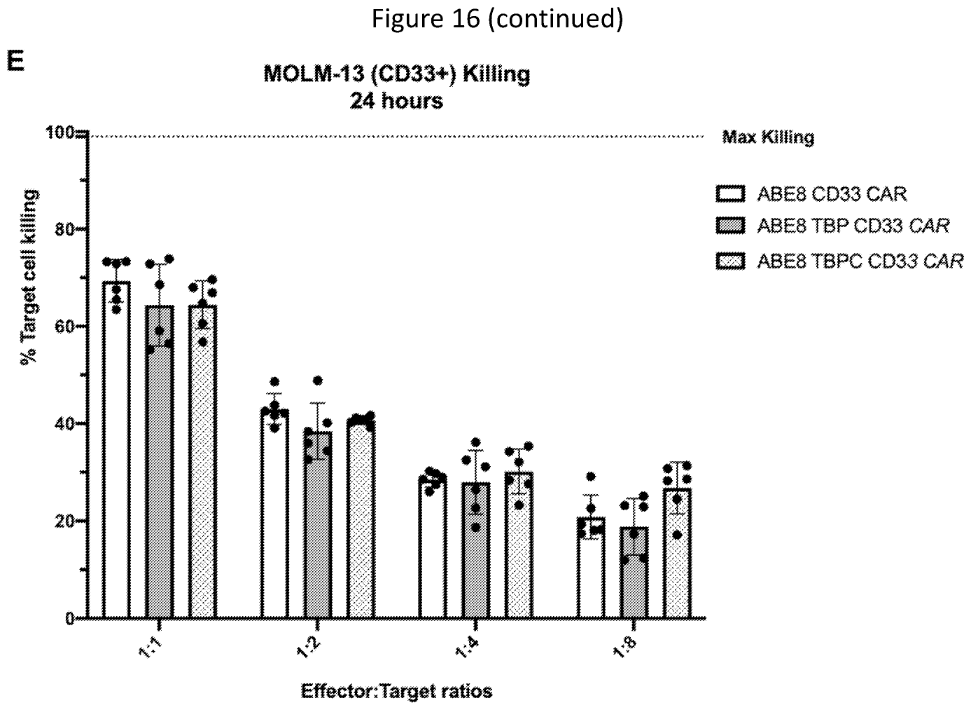

Slight modifications to the base editor retargeting method work-flow increased the transgene integration rate to over 50%/while maintaining incredibly high levels of simultaneous gene knockout (FIG. 15). In lieu of genetic testing, the engineered T cells produced by these methods were subjected to a functional analysis. This analysis demonstrated the ability of T cells that were engineered to express a CAR (i.e., CAR T cells) to kill CAR-antigen positive tumor cells (FIG. 16). Importantly the engineered CAR T cells killed cancer cells with similar efficiencies regardless of how many concurrent gene edits they were subjected to. The results of this experiment demonstrate that the CAR T cells engineered using the retargeting method are functional, are reactive to antigens expressed on target cells, and express IFNγ in response to activation.

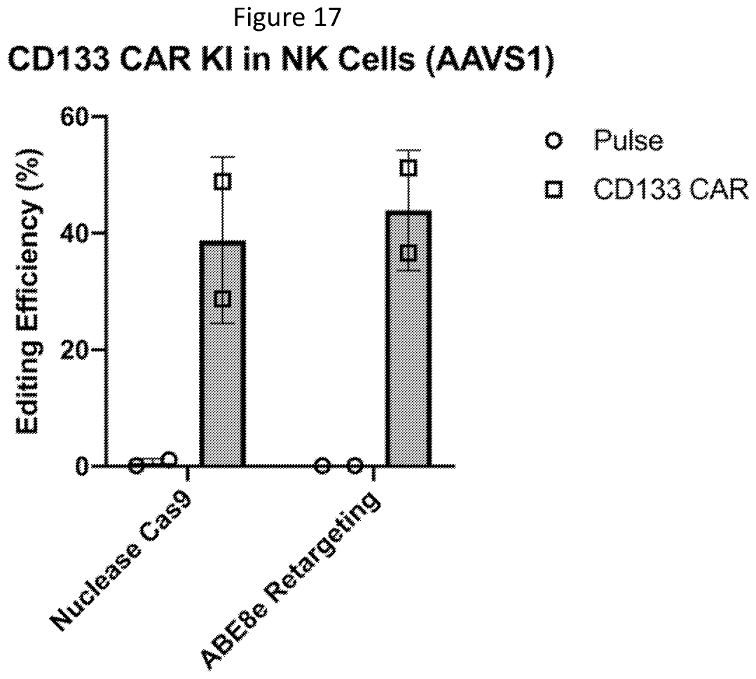

Finally, the base editor retargeting method was used to introduce a CD133-CAR transgene into natural killer (NK) cells (FIG. 17). The results of this experiment reveal that this method can be used to efficiently edit primary human immune cells other than T cells, suggesting that this method may be broadly applicable to a wide variety of clinically relevant human cell products.

### Materials & Methods:

### Coculture and Intracellular Staining

T cells were thawed and cocultured in RPMI1640 medium supplemented with 10% FBS and 1× Pen/Strep with the indicated target cells at a 1:1 effector:target (E:T) ratio at 37° C. with 5% CO₂ for 1 hour before treatment with brefeldin A (10 μg/ml; BD Biosciences) and monensin (0.7 μg/ml; BD Biosciences). The cells were incubated at 37° C. with 5% CO₂ for 12 hours, harvested, washed twice with 1×PBS and incubated with Fixable Viability Dye eFluor780 (eBioscience) for 10 minutes at room temperature. The cells were then washed with 1×PBS and stained with fluorescently labeled antibodies against CD4 and CD8 for 15 minutes at room temperature. The cells were then washed again with 1×PBS permeabilized with Fix/Perm (BD Biosciences) for 20 mins at room temperature, washed with Perm/Wash (BD Biosciences), and were then stained with fluorescently labeled antibodies against IFNγ for 20 minutes at room temperature. The cells were then fixed in 1% PFA and analyzed using a CytoFlex S flow cytometer (Beckman Coulter). Data analysis was performed using FlowJo version 10.6.1 (FlowJo LLC).

### In Vitro CAR-T Cytotoxicity Assay

T cells were thawed and cocultured in RPMI1640 medium supplemented with 10% FBS and 1× Pen/Strep with luciferase-expressing target cells at 1:1, 1:2, 1:4, and 1:8 E:T ratios in triplicate at 37° C. with 5% CO₂ for the indicted times. Target cells alone and Triton-X-100 treated target cells were used as negative and positive controls for targeted killing, respectively. At 24 hours, 5.6 μg of D-luciferin (Goldbio) was added to each sample and bioluminescence was immediately assessed using a BioTek Synergy 2 plate reader running Gen5 software (version 2.04) with an integration time of 1 second. Cell killing was calculated as a percentage of the no-effector control for each E:T ratio.

### Example 2

In the following example, the inventors provide the protocol that was used to introduce a CD133-CAR transgene into natural killer (NK) cells (FIG. 17).

### Materials:

1. Stimmed NK cells: 300K/sample for 10 μL zap, 3e6/sample for 100 μL zap
2. RNA: Cas9 mRNA, ABE8 mRNA, AAVS1 sgRNAs, AAVS1 Nick sgRNA, AAVS1 retargeting 1 sgRNA, and AAVS1 retargeting 2 sgRNA
3. Viral constructs: rAAV MND CD133 Gen2 CAR RQR8, and rAAV MND CD133 Gen4v2 CAR RQR8
4. RNase inhibitor (Millipore Sigma Cat #3335402001)
5. IDT Cas9 Electroporation Enhancer
6. 24-well plate (for 10 μL zap) and 6-well plate (100 μL zap)
7. Neon Transfection Kit (10 μL and 100 μL)
8. NK recovery media (AIM-V+ICSR+100 IU/mL IL-2, no Pen/Strep)

### Protocol:

1. Prepare plates with medium for pre-warm
   a. 24-well plate w/580 μL NK recovery media (if using 10 μL tips, but twice) 6-well plate with 2.9 mL NK recovery media (if using 100 μL tips)
   b. Plate layout:

| | | | |
|---|---|---|---|
| LP#12 Cas9 | LP#12 ABE8e | LP#13 Cas9 | LP#13 ABE8e |
| Ctrl | Ctrl | Ctrl | Ctrl |
| LP#12 Cas9 | LP#12 ABE8e | LP#13 Cas9 | LP#13 ABE8e |
| Gen2 CAR | retarget sgRNAs | Gen2 CAR | retarget sgRNAs |
| | Gen2 CAR | | Gen2 CAR |
| LP#12 Cas9 | LP#12 ABE8e | LP#13 Cas9 | LP#13 ABE8e |
| Gen4v2 CAR | retarget sgRNAs | Gen4v2 CAR | retarget sgRNAs |
| | Gen4v2 CAR | | Gen4v2 CAR |

*Depends on the actual experimental design, but do include an ABE8e/Cas9 mRNA only control

2. Count cells
3. Transfer all the NK cells needed to a 15 mL conical tube
4. Spin 400×g for 5 minutes and pour off supernatant
5. Wash cells with PBS without Ca²⁺ or Mg²⁺, spin at 400×g for 5 minutes and pour off
supernatant
6 Resuspend cells at 8.5 μL/300K (for 10 μL zap) and 85 μL3e6 (for 100 μL zap) in Buffer T
(for primary T and B cells, PBMCs, monocytes, and bone marrow-derived cells) and mix
by pipetting
7. Aliquot cells into sterile microtubes for each transfection
   a. 8.5 μL per sample for 10 μL zap
   b. 85 μL per sample for 100 μL zap
8. Add appropriate nucleic acid to tubes (for ABE8e or Cas9 ctrl, add ABE8e or Cas9 mRNA only)
   a. Cas9/ABE8 (3 μg/μL): 0.5 μL per sample for 10 μL tips; 5 μL for 100 μL tips
   b. sgRNAs:
      i. For Cas9 samples: 1 μL/sample of AAVS1 sgRNA (1 nM) for 10 μL tips; 10 μL/sample of AAVS1 sgRNA (1 nN) for 100 μL tips
      ii. For ABE8e samples: 0.25 μL of each sgRNA (sgRNA all at 4 nM), that's 1 μL total/sample for 10 μL tips; 2.5 μL of each sgRNA (sgRNA all at 4 nM), that's 10 μL total/sample for 100 μL tips
   c. RNase inhibitor: 1 μL of 1:5 diluted solution from stock for 10 μL tips; 10 μL of 1:5 diluted solution from stock for 100 μL tips (incubate at RT for 5 mins)
   d. Electroporation enhancer: 1 μL of 1:5 diluted solution from stock for 10 μL tips; 10 μL of 1:5 diluted solution from stock for 100 μL tips
   e. Final volume for 10 μL should be 12 μL (to avoid bubbles), and for 100 μL should be 120 μL (to avoid bubbles). Use T buffer to fill up to these volumes.
9. Place a Neon cuvette into the machine and fill with 3 mL buffer E (10 μL tip) or buffer E2 (100 μL tip)
10. Grab a Neon tip with the Neon pipette and draw up cell/nucleic acid mixture (make sure to mix properly, and check for air bubbles in the tip—this will cause arcing and kill your cells)
11. Proceed with transfection using Neon machine:
   a. For Cas9 samples, use the saved program: pulse voltage 1850V, pulse number 2, pulse width 10 ms.
   b. For ABE8e samples, use the saved program: pulse voltage 1825V, pulse number 2, pulse width 10 ms.
12. Place transfected cells into the wells of pre-warmed medium and incubate at 37° C. for 0.5-1 hrs
13. During the incubation time, prepare the viral construct

| | Virus | |
|---|---|---|
| | (μL)^{Depends on the titer of viruses} | Media |
| Treatments | MOI = 500K | (μL) |
| Ctrls | 0 | 1600 |
| Gen2 | 37.2 | 1561.2 |
| Gen4v2 | 38.8 | 1561.2 |

14. Add virus drop-wise, accordingly (400 μL per well)
15. After 72 hrs, harvest cells for analysis or do an NK expansion to expand cell numbers

We claim:

1. A method for producing genetically modified cells, the method comprising introducing into the cells:
i. a plasmid, mRNA, or protein encoding a base editor;
ii. two primary gRNAs that are complementary to the 5′ and 3′ ends of a knockin target site prior to editing;
iii. two retargeting gRNAs that are complementary to the 5′ and 3′ ends of a knockin target site after they have been edited by complexes comprising the base editor and the primary gRNAs; and
iv. a DNA donor template;
thereby generating cells with a targeted knockin via homology directed repair from the DNA donor template.

2. The method of claim 1, wherein the method further comprises introducing into the cells at least one additional set of reagents that target a different knockin target site, each additional set of gRNAs comprising:
i. two primary gRNAs that are complementary to two knockin target sites prior to editing;
ii. two retargeting gRNAs that are complementary to the two knockin target sites after they have been edited by complexes comprising the base editor and the primary gRNAs; and
iii. a DNA donor template; and
wherein the method generates cells with two or more targeted knockins.

3. The method of claim 1, wherein the method generates cells with a targeted knockin with at least 40% efficiency.

4. The method of claim 1, wherein the method generates cells with a targeted knockout with at least 90% efficiency.

5. The method of claim 1, wherein the rate of indel formation at the knockin target site is less than 10%, preferably less than 7%.

6. A method for producing genetically modified cells, the method comprising introducing into the cells:
i. a plasmid, mRNA, or protein encoding a base editor;
ii. at least two primary gRNAs that are complementary to the 5′ and 3′ ends of a knockin target site prior to editing; and
iii. a DNA donor template;
thereby generating cells with a targeted knockin via homology directed repair from the DNA donor template; and wherein the rate of indel formation at the knockin target site is less than 10%, and is preferably less than 1%, optionally undetectable.

7. The method of claim 6, wherein the method further comprises introducing into the cells at least one additional set of reagents that target a different knockin target site, each additional set of gRNAs comprising:
i. two primary gRNAs that are complementary to two knockin target sites prior to editing; and
ii. a DNA donor template; and
wherein the method generates cells with two or more targeted knockins.

8. The method of claim 6, wherein the method generates cells with a targeted knockin with at least 20% efficiency.

9. The method of claim 1, wherein the method further comprises introducing into the cells: one or more primary gRNAs that are complementary to a knockout target site prior to editing, and wherein the method generates cells with at least one targeted knockin and at least one targeted knockout.

10. The method of claim 9, wherein the targeted knockout site is in a gene that encodes an alloreactive or immunomodulatory protein.

11. The method of claim 10 wherein the knockout target site is in a gene selected from TRAC, PDCD1, B2M, and CISH.

12. (canceled)

13. The method of claim 1, wherein the base editor is BE3, BE4, or ABE8e.

14. The method of claim 13, wherein the base editor is ABE8e.

15. The method of claim 1, wherein the cells are lymphohematopoietic cells.

16. The method of claim 15, wherein the cells are T cells or natural killer (NK) cells.

17. The method of claim 15, wherein the cells are pluripotent stem cells or progenitor cells capable of differentiating into T cells or NK cells.

18. The method of claim 1, wherein the DNA donor template is provided via a recombinant adeno-associated virus (rAAV).

19. The method of claim 1, wherein the DNA donor template encodes a chimeric antigen receptor (CAR).

20. (canceled)

21. The method of claim 1, wherein the DNA donor template encodes a T cell receptor (TCR).

22. (canceled)

23. (canceled)

24. (canceled)

25. A genetically modified cell obtained according to the method of claim 1.

26. (canceled)


SEQUENCE LISTING (38 sequences)

SEQ ID NO: 1 - DNA, 20 bp, Artificial Sequence
  Synthetic - gRNA for knockin at AAVS1 locus
gtcaccaatcctgtccctag

SEQ ID NO: 2 - DNA, 20 bp, Artificial Sequence
  Synthetic - gRNA for knockin at AAVS1 locus
gtcgccggtcctgtccctag

SEQ ID NO: 3 - DNA, 20 bp, Artificial Sequence
  Synthetic - gRNA for knockin at AAVS1 locus
gttattaatcctgtccctag

SEQ ID NO: 4 - DNA, 20 bp, Artificial Sequence
  Synthetic - gRNA for knockin at AAVS1 locus
cttcctagtctcctgatatt

SEQ ID NO: 5 - DNA, 20 bp, Artificial Sequence
  Synthetic - gRNA for knockin at AAVS1 locus
cttcctggtctcctgatatt

SEQ ID NO: 6 - DNA, 20 bp, Artificial Sequence
  Synthetic - gRNA for knockin at AAVS1 locus
ctttttagtctcctgatatt

SEQ ID NO: 7 - DNA, 20 bp, Artificial Sequence
  Synthetic - gRNA for knockout of PDCD1
cacctacctaagaaccatcc

SEQ ID NO: 8 - DNA, 20 bp, Artificial Sequence
  Synthetic - gRNA for knockout of PDCD1
ggagtctgagagatggagag

SEQ ID NO: 9 - DNA, 20 bp, Artificial Sequence
  Synthetic - gRNA for knockout of PDCD1
ttctctctggaagggcacaa

SEQ ID NO: 10 - DNA, 20 bp, Artificial Sequence
  Synthetic - gRNA for knockout of PDCD1
gacgttacctcgtgcggccc

SEQ ID NO: 11 - DNA, 20 bp, Artificial Sequence
  Synthetic - gRNA for knockout of PDCD1
cctgcagagaaacacacttg

SEQ ID NO: 12 - DNA, 20 bp, Artificial Sequence
  Synthetic - gRNA for knockout of PDCD1
ggggttccagggcctgtctg

SEQ ID NO: 13 - DNA, 20 bp, Artificial Sequence
  Synthetic - gRNA for knockout of PDCD1
cagttccaaaccctggtggt

SEQ ID NO: 14 - DNA, 20 bp, Artificial Sequence
  Synthetic - gRNA for knockout of PDCD1
ggacccagactagcagcacc

SEQ ID NO: 15 - DNA, 20 bp, Artificial Sequence
  Synthetic - gRNA for knockout of TRAC
cttacctgggctggggaaga

SEQ ID NO: 16 - DNA, 20 bp, Artificial Sequence
  Synthetic - gRNA for knockout of TRAC
ttcgtatctgtaaaaccaag

SEQ ID NO: 17 - DNA, 20 bp, Artificial Sequence
  Synthetic - gRNA for knockout of TRAC
tttcaaaacctgtcagtgat

SEQ ID NO: 18 - DNA, 20 bp, Artificial Sequence
  Synthetic - gRNA for knockout of TRAC
ttcaaaacctgtcagtgatt

SEQ ID NO: 19 - DNA, 20 bp, Artificial Sequence
  Synthetic - gRNA for knockout of B2M
actcacgctggatagcctcc

SEQ ID NO: 20 - DNA, 20 bp, Artificial Sequence
  Synthetic - gRNA for knockout of B2M
tcgatctatgaaaaagacag

SEQ ID NO: 21 - DNA, 20 bp, Artificial Sequence
  Synthetic - gRNA for knockout of B2M
cttaccccacttaactatct

SEQ ID NO: 22 - DNA, 20 bp, Artificial Sequence
  Synthetic - gRNA for knockout of CD3E
actcacctgataagaggcag

SEQ ID NO: 23 - DNA, 20 bp, Artificial Sequence
  Synthetic - gRNA for knockout of PDCD1
cacctacctaagaaccatcc

SEQ ID NO: 24 - DNA, 20 bp, Artificial Sequence
  Synthetic - gRNA for knockout of B2M
cttaccccacttaactatct

SEQ ID NO: 25 - DNA, 20 bp, Artificial Sequence
  Synthetic - gRNA for knockout of CISH
ctcaccagattcccgaaggt

SEQ ID NO: 26 - DNA, 3004 bp, Artificial Sequence
  Synthetic - DNA donor template encoding GFP
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcgatcgggtacatcgatcgcaggcgcaatcttcgcatttcttttttccagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcc

SEQ ID NO: 27 - DNA, 82 bp, Artificial Sequence
  Synthetic- Top strand of AAV1 locus - unedited
ggggccactagggacaggattggtgacagaaaagccccatccttaggcctcctccttcctagtctcctgatattgggtctaa

SEQ ID NO: 28 - DNA, 82 bp, Artificial Sequence
  Synthetic- Bottom strand of AAV1 locus - unedited
ccccggtgatccctgtcctaaccactgtcttttcggggtaggaatccggaggaggaaggatcagaggactataacccagatt

SEQ ID NO: 29 - DNA, 82 bp, Artificial Sequence
  Synthetic- Top strand of AAV1 locus - CBE edited
ggggccactagggacaggattggtgacagaaaagccccatccttaggcctcctcctttttagtctcctgatattgggtctaa

SEQ ID NO: 30 - DNA, 82 bp, Artificial Sequence
  Synthetic- Bottom strand of AAV1 locus - CBE edited
ccccggtgatccctgtcctaattactgtcttttcggggtaggaatccggaggaggaaggatcagaggactataacccagatt

SEQ ID NO: 31 - DNA, 82 bp, Artificial Sequence
  Synthetic- Top strand of AAV1 locus - ABE edited
ggggccactagggacaggattggtgacagaaaagccccatccttaggcctcctccttcctggtctcctgatattgggtctaa

SEQ ID NO: 32 - DNA, 82 bp, Artificial Sequence
  Synthetic- Bottom strand of AAV1 locus - ABE edited
ccccggtgatccctgtcctggccgctgtcttttcggggtaggaatccggaggaggaaggatcagaggactataacccagatt

SEQ ID NO: 33 - DNA, 20 bp, Artificial Sequence
  Synthetic - gRNA for knockin at TRAC locus
agaggatcagggttaggaca

SEQ ID NO: 34 - DNA, 20 bp, Artificial Sequence
  Synthetic - gRNA for knockin at TRAC locus
tcagggttctggatatctgt

SEQ ID NO: 35 - DNA, 20 bp, Artificial Sequence
  Synthetic - gRNA for knockin at TRAC locus
gctggtacacggcagggtca

SEQ ID NO: 36 - DNA, 20 bp, Artificial Sequence
  Synthetic - gRNA for knockin at TRAC locus
ccagaaccctgaccctgccg

SEQ ID NO: 37 - DNA, 20 bp, Artificial Sequence
  Synthetic - gRNA for knockin at TRAC locus
cctgccgtgtaccagctgag

SEQ ID NO: 38 - DNA, 20 bp, Artificial Sequence
  Synthetic - gRNA for knockin at TRAC locus
aagtctgtctgcctattcac